(19) United States
(12) Patent Application Publication
(10) Pub. No.: US 2021/0401938 A1
(43) Pub. Date: Dec. 30, 2021

(54) METHODS FOR TREATING CARDIAC INJURY

(71) Applicant: Douglas B. Sawyer, Scarborough, ME (US)

(72) Inventor: Douglas B. Sawyer, Scarborough, ME (US)

(21) Appl. No.: 17/248,217

(22) Filed: Jan. 14, 2021

Related U.S. Application Data
(63) Continuation of application No. 15/762,271, filed on Mar. 22, 2018, now abandoned.
(60) Provisional application No. 62/233,148, filed on Sep. 25, 2015.

(51) Int. Cl. A61K 38/18; G01N 33/50; G01N 33/68; A61K 31/706; A61K 35/34; C07K 14/475

(52) U.S. Cl. CPC A61K 38/1883; G01N 33/5073; G01N 33/6893; A61K 31/706; G01N 2800/52; C07K 14/4756; G01N 2333/4756; G01N 2800/32; A61K 35/34

(57) ABSTRACT

Provided herein are methods for promoting differentiation of cardiac progenitor cells toward myocytes and suppressing the conversion of cardiac progenitor cells into fibroblasts and myofibroblasts cells in a subject determined to have cardiac progenitor cells in or around the heart, by administering a therapeutically effective amount of an NRG-1 peptide or functional variant or fragment thereof. The methods disclosed herein can be used to treat, prevent, or delay the progression of cardiac injury, for example heart failure or myocardial infarction.

### RELATED APPLICATIONS

The present application claims the benefit of priority of U.S. Provisional Patent Application No. 62/233,148, filed Sep. 25, 2015, the contents of which are hereby incorporated by reference herein its entirety.

### STATEMENT AS TO FEDERALLY SPONSORED RESEARCH

This work was partially supported by the National Institutes of Health/National Heart Lung and Blood Institute under Grant U01 HL 100398. Thus, the government has certain rights in the invention.

The disclosure relates to methods of promoting differentiation of cardiac progenitor cells toward myocytes and suppressing the conversion of cardiac progenitor cells into fibroblasts and myofibroblasts by administering a therapeutically effective amount of a neuregulin (NRG) peptide or a functional fragment thereof.

Cardiovascular disease continues to be a leading cause of mortality and morbidity worldwide, accounting for 17.3 million deaths per year. Progressive damage to heart tissue by, e.g., ischemic heart disease, hypertension, diabetes, valvular disease, myocarditis, infections, systemic toxins, and cardiotoxic drugs can ultimately lead to heart failure. There are several compensatory mechanisms that occur as the failing heart attempts to maintain adequate function. These include increasing cardiac output, increasing ventricular volume and wall thickness through ventricular remodeling, and maintaining tissue perfusion with augmented mean arterial pressure through activation of neurohormonal systems. Although initially beneficial in the early stages of heart failure, all of these compensatory mechanisms eventually lead to a vicious cycle of worsening heart failure.

Furthermore, cardiac injury entails complex structural remodeling involving rearrangement of muscle fibers, interstitial fibrosis, accumulation of extracellular matrix, and angiogenesis. Many of the processes underlying cardiac remodeling have features in common with chronic inflammatory processes. During these processes, non-myocyte cells, such as endothelial cells, fibroblasts, and immune cells, residing in or infiltrating into the myocardial interstitium play active roles.

Although new therapies have improved subject outcomes, symptomatic heart failure is still a chronically progressive disease that is not adequately treated with current therapies. Stem cell therapies have emerged as a potential new mechanism for treating severe cardiomyopathy. However, use of human embryonic stem cells has significant limitations in part due to inefficient cardiomyogenic differentiation. Endogenous cardiac stem cells, capable of proliferating and differentiating into cardiac myocytes, have more recently been isolated using stem cell markers such as c-Kit and stem cell antigen-1 (Sca-1). Unfortunately, these cardiac progenitor cells also differentiate into multiple lineages, including myofibroblasts, which are associated with pathogenic cardiac restructuring that can lead to heart failure.

Previously, animal studies and ongoing clinical trials have demonstrated beneficial effects of recombinant NRG as a potential therapeutic on cardiac function (see, e.g., Sawyer et al., 2011; Lenihan et al., 2013). NRG/ErbB signaling has now been found to play an important role in cardiac structural and functional integrity by inhibiting the differentiation of cardiac progenitor cells to myofibroblasts and increasing the differentiation to myocytes. Thus, this invention is based in part on research establishing that ErbB2 and ErbB3 receptors on cardiac progenitor cells are targets for NRG that result in NRG/ErbB signaling that drives differentiation of the cardiac progenitor cells toward formation of myocytes and inhibits differentiation into fibroblasts and myofibroblasts. This finding can be used to identify patients who will benefit most from treatment of cardiac injury, including heart failure, with an NRG peptide or functional variant or fragment thereof.

Thus, one aspect of the invention provides a method for identifying subjects who harbor cardiac progenitor cells that are responsive to treatment with NRG. This may be determined by a biopsy of the heart tissue obtained, e.g., during cardiac surgery, or cardiac catheterization. The method may include the step of isolating the cells from the subject or may be carried out entirely in vitro using a sample of cells originating from the subject. Cardiac progenitor cells isolated from the biopsy material may then be exposed to an NRG peptide or functional variant or fragment thereof to determine whether the cells respond by exhibiting reduced conversion to fibroblasts and myofibroblasts and/or by preferentially differentiating into cardiac myocytes. Subjects whose cardiac progenitor cells demonstrate this response to the NRG peptide or functional variant or fragment thereof can be expected to respond well to treatment for or prevention of cardiac injury with an NRG peptide or functional fragment or variant thereof.

Thus, another aspect of the invention provides methods for treating a subject found to harbor cardiac progenitor cells that respond to an NRG peptide or functional variant or fragment thereof by preferential differentiation away from fibroblasts and/or toward cardiac myocytes. The invention also provides an NRG peptide or functional variant or fragment thereof for use in a method of treating such a subject, for example in a method of treating or preventing cardiac injury, a method of inducing cardiac tissue regeneration or a method of repairing cardiac tissue. The methods may be carried out in any subject that has been identified by a method as described herein. The methods comprise administering a therapeutically effective amount of an NRG peptide or a functional variant or fragment thereof to promote differentiation of cardiac progenitor cells towards cardiac monocytes and suppress the conversion of cardiac progenitor cells into fibroblasts and myofibroblasts in a subject with cardiac injury. Other aspects of the invention provide methods for inducing formation of cardiac tissue, strengthening cardiac tissue, and preventing the onset of cardiac injury by administering a therapeutically effective amount of a NRG peptide or functional variant or fragment thereof to a patient identified as having cardiac progenitor cells responsive to NRG. Also provided are an NRG peptide or functional variant or fragment thereof for use in a method of inducing formation of cardiac tissue, strengthening cardiac tissue, or preventing the onset of cardiac injury by administering a therapeutically effective amount of a NRG peptide or functional variant or fragment thereof to a patient identified as having cardiac progenitor cells responsive to NRG. Activation of NRG/ErbB signaling by administering an NRG peptide or functional variant or fragment thereof to a subject promotes differentiation of cardiac progenitor cells into cardiac myocytes in those subjects, leading to enhanced myocardial regeneration and improved heart function. The methods of the invention may comprise co-administration of human embryonic stem cells or human cardiac progenitor cells.

In certain embodiments, suppressing the conversion of cardiac progenitor cells into fibroblast and myofibroblast cells induces cardiac tissue regeneration by driving differentiation toward myocyte formation. In other embodiments, suppressing the conversion of cardiac progenitor cells into fibroblast and myofibroblast cells and/or inducing differentiation into cardiac myocyte cells, repairs and strengthens cardiac tissue. In another embodiment, suppressing the conversion of cardiac progenitor cells into fibroblast and myofibroblast cells prevents cardiac fibrosis. In more specific embodiments, decreasing cardiac fibrosis after cardiac injury prevents formation of scar tissue. In some embodiments, scar tissue may be reversed or repaired. In certain embodiments, suppressing the conversion of cardiac progenitor cells into fibroblasts and myofibroblasts cells prevents the onset of cardiac injury.

In one aspect, the methods of inducing cardiac tissue regeneration after cardiac injury in a subject found to have cardiac progenitor cells that are responsive to treatment with NRG comprise administering a therapeutically effective amount of an NRG peptide or a functional variant or fragment thereof.

In another aspect, the methods of repairing cardiac tissue after cardiac injury in a subject found to have cardiac progenitor cells that are responsive to treatment with NRG comprise administering a therapeutically effective amount of an NRG peptide or a functional variant or fragment thereof.

In a further aspect, the methods of preventing the onset of cardiac injury in a subject found to have cardiac progenitor cells that are responsive to treatment with NRG comprise administering a therapeutically effective amount of an NRG peptide or a functional variant or fragment thereof.

In certain embodiments, the cardiac injury results from a cardiovascular disease. In some embodiments, the cardiovascular disease results from coronary artery disease, stroke, myocardial infarction, cardiomyopathy, hypertension, ischemic heart disease, atrial fibrillation, congenital heart disease, myocarditis, endocarditis, pericarditis, atherosclerosis, vascular disease, coronary bypass surgery, exposure to a cardiotoxic compound, thyroid disease, viral infection, gingivitis, drug abuse, alcohol abuse, or high blood cholesterol. In specific embodiments, the subject has left ventricular systolic dysfunction. In some embodiments, the subject is suffering from heart failure.

In certain embodiments, the subject is at risk of developing a cardiac injury or has a history of cardiovascular disease. In other embodiments, the subject may be contemplating chemotherapy with a chemotherapeutic agent known to damage cardiac tissue. Identifying whether the subject has cardiac progenitor cells that are responsive to treatment with NRG will indicate that the subject will benefit from concurrent therapy with an NRG peptide or functional variant or fragment thereof. Thus, in some embodiments, the methods provided herein further comprise administering a therapeutically effective amount of an anticancer agent, e.g, azacitidine before, during or after administration of the NRG peptide or functional variant or fragment thereof.

In other embodiments, provided herein the subject is a mammal. In some embodiments, the mammal is a human. In some embodiments, the human is suffering from a cancer and is already receiving cancer treatment that has been associated with damage to cardiac tissue and function. In some embodiments the human is a child or infant suffering from a congenital heart injury or recovering from heart surgery, particularly surgery to remodel or redesign portions of the heart.

In some embodiments, the NRG peptide or functional variant or fragment thereof is administered intravenously or subcutaneously.

In certain embodiments, the NRG peptide is an NRG-1 or an NRG-2 peptide, (particularly, an NRG-1β, and more particularly, Glial Growth Factor (GGF) 2 peptide (e.g., Cimaglermin alpha) or a functional variant or fragment thereof. In some specific embodiments, NRG peptide comprises the amino acid sequence of SEQ ID NO:1, or a functional variant or fragment of SEQ ID NO:1. In other specific embodiments the NRG peptide comprises the amino acid sequence SEQ ID NO:2, or a functional fragment of SEQ ID NO:2. In some embodiments, the NRG peptide comprises the amino acid sequence of SEQ ID NO:21 or SEQ ID NO:22, or a functional fragment thereof.

In certain embodiments, the NRG peptide or functional variant or fragment thereof binds to ErbB3 receptors expressed on cardiac progenitor cells to activate ErbB signaling.

In certain embodiments, binding of the NRG peptide or functional variant or fragment thereof to ErbB3 receptors activates ErbB signaling. In at least some embodiments, the NRG peptide or functional variant or fragment thereof binds to ErbB3 receptors and effects ErbB signaling by recruiting ErbB2 receptors on the cardiac progenitor cells. The resulting ErbB signaling promotes differentiation of cardiac progenitor cells toward cardiac myocytes and/or suppresses the conversion of cardiac progenitor cells into fibroblasts and myofibroblasts.

Another aspect of the invention provides a method of identifying a subject who will benefit from treatment or prevention of cardiac injury with NRG comprising:
a) isolating human cardiac progenitor cells from a subject with cardiac injury or at risk of cardiac injury;
b) exposing the subject's cells to an NRG peptide or functional variant or fragment;
c) evaluating whether the cells are responsive to NRG by determining whether conversion of cells into fibroblasts and myofibroblasts is suppressed and/or whether the cells preferentially differentiate into cardiac myocytes; and
   wherein if suppression of the conversion of the cardiac progenitor cells into fibroblasts and myofibroblasts or preferential differentiation into cardiac myocytes are found, then the subject will benefit from treatment or prevention of cardiac injury with the NRG peptide or functional variant or fragment thereof. In a related aspect, the method is carried out on cardiac progenitor cells from the subject, and the method does not include a step of isolating those cells. For example, provided is a method of identifying a subject who will benefit from treatment or prevention of cardiac injury with NRG comprising:
a) exposing human cardiac progenitor cells from a subject with cardiac injury or at risk of cardiac injury, to an NRG peptide or functional variant or fragment in vitro; and
b) evaluating whether the cells are responsive to neuregulin by determining whether conversion of cells into fibroblasts and myofibroblasts is suppressed and/or whether the cells preferentially differentiate into cardiac myocytes;
   wherein if suppression of the conversion of the cardiac progenitor cells into fibroblasts and myofibroblasts or preferential differentiation into cardiac myocytes are found, then the subject will benefit from treatment or prevention of cardiac injury with the NRG peptide or functional variant or fragment thereof

Another aspect of the invention provides a method of treating or preventing cardiac injury comprising:
a) isolating human cardiac progenitor cells from a subject with cardiac injury or at risk of cardiac injury;
b) evaluating whether the subject's cardiac progenitor cells respond to an NRG peptide or functional variant or fragment thereof by suppressing the conversion of cardiac progenitor cells into fibroblasts and myofibroblasts and/or promoting differentiation of the cardiac progenitor cells into cardiac myocytes; and
c) if the cardiac progenitor cells are responsive, administering NRG peptide or functional variant or fragment thereof to treat or prevent or reduce the severity of cardiac injury.
   In a related aspect, the invention provides an NRG peptide or functional variant or fragment thereof for use in such a method, or for use in a method of treating a subject who has been identified by a method as described herein as being a subject who will benefit from treatment or prevention of cardiac injury.

In another embodiment, the methods of the invention comprise
a) culturing and expanding cardiac progenitor cells found to be capable of responding to NRG peptide or functional variant or fragment thereof by suppressing the conversion of cardiac progenitor cells into fibroblasts and myofibroblasts and/or promoting differentiation of the cardiac progenitor cells into cardiac myocytes; and
b) administering the expanded cardiac progenitor cells to the subject with a therapeutically effective amount of an NRG peptide or functional variant or fragment thereof. Also provided is an NRG peptide or functional variant or fragment thereof for use in such a method

Another aspect of the invention provides a method of producing a cell population enriched in cardiac myocytes, comprising:
a) isolating the cardiac progenitor cells obtained from a subject;
b) culturing the cells in a growth medium;
c) incubating the cells with an effective amount of an NRG peptide or a functional variant or fragment thereof to promote differentiation to cardiac myocytes; and
d) isolating the myocytes.
   This method may be followed by administration of the cardiac myocytes to the subject. The cells may be isolated in step a) from a sample in vitro, such as a biopsy sample as described herein.

### BRIEF DESCRIPTION OF DRAWINGS

FIG. 1A, FIG. 1B, and FIG. 1C show that murine Sca-1^{pos}CD31^{neg} cardiac progenitor cells express ErbB2 and ErbB3 receptors. FIG. 1A depicts representative flow cytometric histograms that demonstrate the purity of an isolated subpopulation of cardiac progenitor cells before- (left panel) and after- (right panel) magnetic activation cell sorting. FIG. 1B shows a profile of mRNA expression of ErbB receptors in cardiac progenitor cells. FIG. 1C depicts representative flow cytometry histograms of cell surface markers on Sca-1^{pos}/CD31^{neg} cardiac progenitor cells. The shaded areas represent the fluorescence of cells treated with corresponding isotype-matching antibody controls.

FIG. 2A, FIG. 2B, and FIG. 2C show that murine Sca-1^{pos}CD31^{neg} cardiac progenitor cells differentiate towards endothelial cells and cardiac myocytes in vitro. FIG. 2A depicts exemplary micrographs of capillary-like structure formation after incubation of cardiac progenitor cells in growth medium (left panel) or endothelial cell (EC)-differentiating media (middle panel). CD31^{pos} cardiac endothelial cells were used as a positive control (right panel). FIG. 2B shows a graphical representation of morphogenic activity of cardiac progenitor cells, incubated in growth medium (control, left bar) or in endothelial cells differentiating media (diff, middle bar), and cardiac endothelial cells (EC, right bar). Capillary tube formation was estimated by measuring their total length. FIG. 2C illustrates and exemplary real-time-PCR analysis of cardiac-specific gene expression in cardiac Sca-1^{pos}CD31^{neg} cells cultured in normal growth medium (Con) or in cardiac myocyte-differentiating media (Diff) for 1 or 3 weeks (w). The values are averages of three experiments. cTnT, cardiac troponin T; -MHC, -myosin heavy chains.

FIG. 3A, FIG. 3B, FIG. 3C, and FIG. 3D show that NRG-1 prevents transition of murine cardiac progenitor cells into myofibrobasts. FIG. 3A depicts representative flow cytometric dot plots that demonstrate there is an accumulation of cardiac progenitor cells towards αSMA positive and collagen 1 producing myofibroblasts in vivo on day 7 after experimental myocardial infarction (D7, MI) in mice. FIG. 3B depicts a graphical representation of data from flow cytometric analysis of αSMA positive (left) and collagen 1α positive (right) Sca-1^{pos}CD31^{neg} cardiac progenitor cells. P values are indicated, unpaired t test. FIG. 3C depicts representative cytofluorographic dot plots showing the expression of αSMA protein in cardiac progenitor cells incubated with TGFβ alone (TGFβ) or in combination with 30 ng/ml NRG-1 (TGFβ+NRG-1) for 48 hrs. FIG. 3D depicts mean fluorescence intensity of αSMA expression in cardiac Sca-1^{pos}CD31^{neg} progenitor cells as assessed by flow cytometry. Data represent mean±SEM from three independent experiments. P-values indicate significance level calculated by t-test.

FIG. 4 shows that ErbB2 and ErbB3 receptors localize in the vascular/peri-vascular regions in the human heart. Green: staining for ErbB2 (left panel) and ErbB3 (right panel) (Abs from Invitrogen and Santa Cruz Biotech, respectively); red: phalloidin; blue: TO-PRO-3 (nuclear staining); yellow arrows indicate peri-vascular staining.

FIG. 5A, FIG. 5B, and FIG. 5C show that NRG-1 prevents transition of human cardiac progenitor cells into myofibrobasts. FIG. 5A depicts phase contrast micrographs of human cardiac progenitor cells immediately after replating a single cell-derived clone (d0, upper panel) and 3 days later (d3, lower panel). Scale bar=100 μm. FIG. 5B depicts real time-PCR analysis of cardiac-specific gene expression in human cardiac progenitor cells before (C) and after culturing in differentiating media for 1 or 2 weeks (w). Values are averages of three experiments, unpaired t test. FIG. 5C depicts flow cytometry histograms of cell surface markers on human cardiac progenitor cells. The shaded areas represent the fluorescence of cells treated with corresponding isotype-matching antibody controls

### EXEMPLARY EMBODIMENTS

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “a” entity or “an” entity refers to one or more of that entity. For example, reference to “a peptide” includes a mixture of two or more such peptides, and the like. As such, the terms “a”, “an”, “one or more” and “at least one” can be used interchangeably. For example, “a dose” includes one or more doses. Further, unless otherwise required by context, singular terms shall include pluralities and plural terms shall include the singular.

As used herein, the term “about” is a stated value plus or minus another amount; thereby establishing a range of values. In certain preferred embodiments “about” indicates a range relative to a base (or core or reference) value or amount plus or minus up to 15%, 14%, 13%, 12%, 11%, 10%, 9%, 8%, 7%, 6%, 5%, 4%, 3%, 2%, 1%, 0.75%, 0.5%, 0.25% or 0.1%.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

As used herein, the term “cardiotoxic” refers to a compound that decreases heart function by directly or indirectly impairing or killing cardiomyocytes.

As used herein, the term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

As used herein the term “intermittent or discontinuous administration” includes a regimen for dosing on intervals of at least (or not less than) 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof so long as the interval/regimen is at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer. For example, the peptide is administered on a dosing interval for at least 2 weeks, e.g., at least 2 weeks, 3 weeks, or 4 weeks. For example, the dosing interval is greater than 4 months.

As used herein, the term “NRG peptide” refers to a peptide that binds to at least ErbB3 on cardiac progenitor cells and activates ErbB signaling. NRG peptides include NRG-1, NRG-2, or an epidermal growth factor (EGF)-like domain containing peptide that binds to at least the ErbB3 receptor and recruits the ErbB2 receptor to effect ErbB signaling. An “EGF-like domain containing peptide” bears a structural similarity to the EGF receptor-binding domain, e.g., as disclosed in Holmes et al., 1992; U.S. Pat. Nos. 5,530,109; 5,716,930; 7,037,888; Hijazi et al., 1998; Chang et al., 1997; Carraway et al., 1997; Higashiyama et al., 1997; and WO 97/09425. NRG-1 peptides are described in U.S. Pat. Nos. 5,530,109; 5,716,930; and 7,037,888, each of which is incorporated herein by reference in its entirety. NRG-2 peptides are described in U.S. Pat. No. 8,114,838 incorporated herein by reference in its entirety. In some embodiments, the NRG-2 peptide is NRG-2α. In some embodiments, the NRG-2 peptide is NRG-2β. In certain embodiments, the NRG peptide employed in the methods of the invention is an NRG-1β peptide, e.g., isoform GGF2 (SEQ ID NO:1 or SEQ ID NO:2) or a functional variant or fragment thereof. In other embodiments the NRG peptide employed in the methods of the invention is an NRG-2 peptide, such as, e.g., NRG-2α (SEQ ID NO:21) or NRG-2β (SEQ ID NO:22), or a functional variant or fragment thereof. The term “NRG peptide or functional variant or fragment thereof” is meant to include an NRG peptide as disclosed herein, a functional variant of an NRG peptide, a functional fragment of an NRG peptide, or a functional fragment of a functional variant of an NRG peptide.

As used herein, the term “functional variant” of an NRG peptide means a peptide that possesses an EGF-like domain and binds to ErbB3, recruits ErbB2, and induces NGR/ErbB signaling leading to suppressed conversion of cardiac progenitor cells to fibroblasts and myofibroblasts and/or by preferential differentiation of cardiac progenitor cells into cardiac myocytes. The functional variant of NRG may bear substantial sequence similarity to GGF2. In some embodiments, the functional variant of an NRG peptide is 80%, 82%, 85%, 88%, 90%, 92%, 95%, 98%, or 99% identical to SEQ ID NO:1, SEQ ID NO:2, SEQ ID NO:21, or SEQ ID NO:22 or a functional fragment thereof. In some embodiments, the variant differs from a corresponding portion of SEQ ID NO:1, SEQ ID NO:2, SEQ ID NO:21, or SEQ ID NO:22 by amino acid substitution, deletion, or insertion. In some embodiments, a variant differs from SEQ ID NO:1, SEQ ID NO:2, SEQ ID NO:21, or SEQ ID NO:22 only by conservative substitution of amino acids. In some embodiments a variant differs from a corresponding portion of SEQ ID NO:1, SEQ ID NO:2, SEQ ID NO:21, or SEQ ID NO:22 by less than 25, less than 20, less than 15, less than, 12, less than 10, less than 8, less than 5, less than 2 amino acid substitutions, which may be conservative substitutions.

As used herein, the term “functional fragment” of an NRG peptide refers to any truncated portion of an NRG peptide, e.g., having the amino acid sequence of SEQ ID NO:1, SEQ ID NO:2, SEQ ID NO:21, or SEQ ID NO:22, or functional variant thereof, that retains the ability to bind to at least ErbB3 on cardiac progenitor cells and activate NRG/ErbB signaling, resulting in suppressed conversion of cardiac progenitor cells to fibroblasts and myofibroblasts and/or by preferential differentiation of cardiac progenitor cells into cardiac myocytes.

As used herein, “responsive to neuregulin” and “responsive to treatment with neuregulin” refers to the cardiac progenitor cells that preferentially differentiate into cardiac myocytes and/or exhibit reduced differentiation into fibroblasts or myofibroblasts upon exposure to an NRG peptide or a functional variant or fragment thereof.

As used herein, the phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial compound. An adjuvant is included under these phrases.

The peptides and functional variants and fragments thereof are purified and/or isolated. As used herein, an “isolated” or “purified” peptide, variant, or fragment, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated peptide is free of the amino acids or sequences that flank it in its naturally-occurring state. Purified also defines a degree of sterility that is safe for administration to a human subject, e.g., lacking infectious or toxic agents.

As used herein, the term “preventing” means minimizing or partially or completely inhibiting the development of fibrosis and scar tissue resulting from cardiac injury.

As used herein, the term “regeneration” refers to the restoration of function to a lost or damaged cell, tissue or organ where function has been compromised. Regeneration capacity can be measured as a function of the cell, tissue, or organ. Such functions can be, but are not limited to expression of proteins, tissue remodeling, induction of angiogenesis/vasculogenesis, reduction in hypertrophy, and coordinated function as tissue or organ, contractility and relaxation. In some embodiments, at least 20, 30, 40, 50, 60, 70, 80, 90, 95, 98, 99 or 100% of the function of the organ is regenerated.

As used herein, the term “steady state levels” refers to a level(s) of an exogenous agent, e.g., a peptide that is sufficient to achieve equilibration (within a range of fluctuation between succeeding doses) between administration and elimination. “Maintaining steady state therapeutic levels” refers to sustaining the concentration of an exogenous agent at a level sufficient to confer therapeutic benefit to a subject.

As used herein, the term “therapeutically effective amount” is intended to mean that amount of a drug or pharmaceutical agent, e.g., an NRG peptide described herein, such as NRG-1β, particularly GGF2, e.g., a peptide having the amino acid sequence of SEQ ID NO:1 or SEQ ID NO:2, or a functional variant or fragment thereof that elicits reduction in the number of myofibroblasts produced and/or increases the number of myocytes produced from endogenous cardiac progenitor cells or co-administered cardiac progenitor cells. A “therapeutically effective amount” is an amount sufficient to improve or maintain the health and integrity of cardiac tissue, decrease or lessen the incidence of symptoms associated with cardiac injury or fibrosis, to normalize body functions in disease or disorder associated with cardiac injury that results in impairment of specific bodily functions, or to provide improvement in one or more of the clinically measured parameters of a disease involving cardiac injury.

As used herein, the term “treating” means that administration of an NRG peptide described herein, such as NRG-1β, particularly isoform GGF2, e.g., a peptide having the amino acid sequence of SEQ ID NO:1 or SEQ ID NO:2, or a functional variant or fragment thereof, will slow or inhibit the progression of cardiac injury that would occur in the absence of treatment, in a statistically significant manner in a subject found to have cardiac progenitor cells that are responsive to NRG. Well known indicia such as left ventricular ejection fraction, exercise performance, mitral valve regurgitation, dyspnea, peripheral edema, and other clinical tests as enumerated above, as well as survival rates and hospitalization rates may be used to assess disease progression. Whether or not a treatment slows or inhibits cardiac injury progression in a statistically significant manner may be determined by methods that are well known in the art (see, e.g., SOLVD Investigators, 1992 and Cohn et al., 1998, incorporated herein by reference).

NRG/ErbB Signaling in Cardiac Progenitor Cells

Normally, fibrosis due to activation of cardiac fibroblasts impedes cardiac regeneration and contributes to loss of contractile function, pathological remodeling and susceptibility to heart failure and myocardial infarction after cardiac injury. It has now been found that the mammalian heart can be stimulated to maximize native regenerative potential following cardiac injury by administering an NRG peptide or functional variant or fragment thereof. In some embodiments, the patient has been identified by previous procedure as having a supply of native cardiac progenitor cells. Alternatively, this native regenerative potential may be supplemented by co-administration (simultaneously or sequentially, continuously or intermittently) of human cardiac progenitor cells or human embryonic stem cells. Ideally, the co-administered cardiac progenitor cells were originally obtained from the subject being treated.

Administration of the NRG peptide or functional variant or fragment thereof promotes differentiation of cardiac progenitor cells toward cardiac myocytes and suppresses the conversion of cardiac progenitor cells into fibroblasts and myofibroblasts in a subject.

In certain embodiments, administering to a subject a therapeutically effective amount of an NRG peptide or functional variant or fragment thereof promotes differentiation of cardiac progenitor cells toward cardiac myocytes and suppresses the conversion of cardiac progenitor cells into fibroblasts and myofibroblasts cells over a period of at least 12 weeks, at least 10 weeks, at least 8 weeks, at least 6 weeks, at least 4 weeks, at least 2 weeks or at least 1 week after administration. In other embodiments, administering to a subject a therapeutically effective amount of an NRG peptide or functional variant or fragment thereof, promotes differentiation of cardiac progenitor cells toward cardiac myocytes and suppresses the conversion of cardiac progenitor cells into fibroblasts and myofibroblasts cells over a period of at least 10 days, at least 9 days, at least 8 days, at least 7 days, at least 6 days, at least 5 days, at least 4 days, at least 3 days, at least 2 days or at least 1 day after administration. In another embodiment, administering to a subject a therapeutically effective amount of an NRG peptide or functional variant or fragment thereof promotes differentiation of cardiac progenitor cells toward cardiac myocytes and suppresses the conversion of cardiac progenitor cells into fibroblasts and myofibroblasts cells for at least 70 hours, at least 60 hours, at least 50 hours, at least 40 hours, at least 30 hours, at least 20 hours, at least 15 hours, at least 10 hours, at least 5 hours, at least 4 hours, at least 3 hours, at least 2 hours, or at least 1 hour after administration.

In another embodiment, administering to a subject an NRG peptide or functional variant or fragment thereof promotes differentiation of at least about 0.1%, 0.2%, 0.3%, 0.4%, 0.5%, 0.6%, 0.7%, 0.8%, 0.9%, 1%, 5%, 10%, 15%, 20%, 30%, 40%, 50%, 60%, 70%, 80%, 90%, 95%, or 100% of cardiac progenitor cells toward cardiac myocytes.

In yet other embodiments, administering to a subject an NRG peptide or functional variant or fragment thereof, suppresses the conversion of cardiac progenitor cells into fibroblasts and myofibroblasts cells by about 1%, 5%, 10%, 15%, 20%, 30%, 40%, 50%, 60%, 70%, 80%, 90%, 95%, or 100%.

In certain embodiments, administering to a subject an NRG peptide or functional variant or fragment thereof, promotes differentiation of at least about 0.1%, 0.2%, 0.3%, 0.4%, 0.5%, 0.6%, 0.7%, 0.8%, 0.9%, 1%, 5%, 10%, 15%, 20%, 30%, 40%, 50%, 60%, 70%, 80%, 90%, 95%, or 100% of cardiac progenitor cells toward cardiac myocytes and suppresses the conversion of cardiac progenitor cells into fibroblasts and myofibroblasts cells by about 1%, 5%, 10%, 15%, 20%, 30%, 40%, 50%, 60%, 70%, 80%, 90%, 95%, or 100%.

In certain embodiments, administering to a subject an NRG peptide or functional variant or fragment thereof results in reduced production of fibroblasts and increased production of functional cardiac myocytes. In other embodiments, administering to a subject an NRG peptide or functional variant or fragment thereof has an anti-fibrotic effect.

In certain embodiments, administering to a subject an NRG peptide or functional variant or fragment thereof, suppresses myocardial fibrosis. In other embodiments, administering to a subject an NRG peptide or functional variant or fragment thereof, reduces expression of pro-fibrotic genes. In specific embodiments, administering to a subject an NRG peptide or functional variant or fragment thereof, reduces the expression of collagens, fibrillins, osteonectin, periostin, and versican.

In certain embodiments, the methods of promoting differentiation of cardiac progenitor cells into cardiac myocytes and suppressing the conversion of cardiac progenitor cells into fibroblasts and myofibroblasts cells in a subject comprise administering to the subject an NRG peptide or functional variant or fragment thereof, and co-administering (simultaneously, sequentially, serially, or intermittently) a vector that expresses a cardiac transcription factor. In certain embodiments, the cardiac transcription factor is GATA4, Hand2, MEF2C, MesP1, Nkx2-5, or Tbx5.

In certain embodiments, the methods of promoting differentiation of cardiac progenitor cells into cardiac myocytes and suppressing the conversion of cardiac progenitor cells into fibroblasts and myofibroblasts cells comprise administering an NRG peptide or functional variant or fragment thereof to a subject suffering from a cancer. The NRG peptide or functional variant or fragment thereof may be administered before, after, or concurrently with a chemotherapeutic agent. It may be administered simultaneously or sequentially with the chemotherapeutic agent. In some embodiments, the chemotherapeutic agent is Herceptin. In some embodiments, the chemotherapeutic agent is selected from bendamustine, busulfan, carmustine, chlorambucil, cyclophosphamide, dacarbazine, ifosfamide, melphalan, procarbazine, streptozocin, temozolomide, asparaginase, capecitabine, cytarabine, 5-Fluoro Uracil, fludarabine, gemcitabine, methotrexate, pemetrexed, raltitrexed, actinomycin D/dactinomycin, bleomycin, daunorubicin, doxorubicin, doxorubicin (pegylated liposomal), epirubicin, idarubicin, mitomycin, mitoxantron, asparaginase, capecitabine, cytarabine, 5-Fluoro Uracil, fludarabine, gemcitabine, methotrexate, pemetrexed, raltitrexed, actinomycin D/Dactinomycin, bleomycin, daunorubicin, doxorubicin, doxorubicin (pegylated liposomal), epirubicin, idarubicin, mitomycin, mitoxantrone, etoposide, docetaxel, irinotecan, paclitaxel, topotecan, vinblastine, vincristine, vinorelbine, carboplatin, cisplatin, oxaliplati, alemtuzamab, BCG, bevacizumab, cetuximab, denosumab, erlotinib, gefitinib, imatinib, interferon, ipilimumab, lapatinib, panitumumab, rituximab, sunitinib, sorafenib, temsirolimus, trastuzumab, clodronate, ibandronic acid, pamidronate, zolendronic acid, anastrozole, abiraterone, amifostine, bexarotene, bicalutamide, buserelin, cyproterone, degarelix, exemestane, flutamide, folinic acid, fulvestrant, goserelin, lanreotide, lenalidomide, letrozole, leuprorelin, medroxyprogesterone, megestrol, mesna, octreotide, stilboestrol, tamoxifen, thalidomide or triptorelin.

In certain embodiments, the methods of promoting differentiation of cardiac progenitor cells into cardiac myocytes and suppressing the conversion of cardiac progenitor cells into fibroblasts and myofibroblasts cells in a subject comprise administering to the subject an NRG peptide or functional variant or fragment thereof, and co-administering (simultaneously, sequentially, continuously, or intermittently) human cardiac progenitor cells that are responsive to NRG. In some embodiments, the human cardiac progenitor cells are initially isolated from the subject receiving treatment and expanded in vitro prior to readmininstration with the NRG peptide or functional variant or fragment thereof. In certain embodiments, the cardiac progenitor cells administered in the methods of the invention express stem cell antigens, c-kit, sca-1, isl-1, SSEA-I or ABCG2. In other embodiments, provided herein the cardiac progenitor cells express cardiac specific markers; e.g. NKx2.5, GATA4, α-MHC. In a preferred embodiment, the cardiac progenitor cells express sca-1. In other embodiments, the cardiac progenitor cells do not express c-kit. In some embodiments, the cardiac progenitor cells maybe cardiospheres.

In certain embodiments, the cardiac progenitor cells provided herein are obtained from and/or administered to the atrial and/or ventricles of the heart. In more specific embodiments, the cardiac progenitor cells are obtained from and/or administered to the left ventricle. In yet more specific embodiments, the cardiac progenitor cells are obtained from and/or administered to the left ventricle free wall, a vascular or a perivascular region of the heart. In certain embodiments, the cardiac progenitor cells obtained from and/or administered express sca-1. Cardiac progenitor cells may be isolated by any means known in the art or disclosed herein.

In certain embodiments, administering to a subject an NRG peptide or functional variant or fragment thereof, limits TGF-β activation and decreases fibroblast activation. TGF-β exists in three isoforms (TGF-β1, TGF-β2, and TGF-β3) that have distinct but overlapping functions in immunity, inflammation, and tissue repair, and TGF-β also has a central role in fibroblast activation and differentiation into myofibroblasts.

Neuregulin

NRGs are growth factors related to the epidermal growth factor superfamily that bind to ErbB receptors. They have been shown to improve cardiac function in multiple models of heart failure, cardiotoxicity and ischemia. NRGs have also been shown to protect the nervous system in models of stroke, spinal cord injury, nerve agent exposure, peripheral nerve damage and chemotoxicity (for review see Sawyer and Caggiano, 2011).

Family members of NRG comprise NRG-1, NRG-2, NRG-3 and NRG-4 genes possess EGF-like domains that allow them to bind to and activate ErbB receptors. Each of the NRG genes can be expressed as multiple distinct protein isoforms due to alternate splicing transcripts (Falls, 2003). NRG also comprises variants or functional homologues with conservative amino acid substitutions that do not substantially alter their biological activity. Suitable conservative substitutions of amino acids are known to those skilled in the art and may be generally made without altering the biological activity of the resulting molecule.

Holmes et al. (, 1992) have shown that the EGF-like domain alone is sufficient to bind and activate the ErbB signaling Accordingly, any peptide product encoded by the NRG-1, NRG-2, NRG-3, or NRG-4 gene, or any NRG-like peptide, e.g., a peptide having an EGF-like domain encoded by a NRG gene or cDNA (e.g., an EGF-like domain containing the NRG-1 peptide subdomains C-C/D or C-C/D′, as described in U.S. Pat. Nos. 5,530,109, 5,716,930, and 7,037,888; or an EGF-like domain as disclosed in WO 97/09425) can be used in the methods of the disclosure to prevent, treat, or delay the progression of cardiovascular disease, e.g., heart failure. The contents of each of U.S. Pat. Nos. 5,530,109; 5,716,930; 7,037,888; and WO 97/09425 are incorporated herein in its entirety.

NRG-1 comprises a group of approximately 15 distinct structurally-related isoforms (Lemke, 1996; Peles and Yarden, 1993). In some embodiments the peptide or functional fragment thereof used in the methods of the disclosure comprises at least 1, at least 2, at least 3, at least 4, at least 5, at least 6, at least 7, at least 8, at least 9, at least 10, at least 11, at least 12, at least 13, at least 14, or at least 15 NRG-1 isoforms. These isoforms can be divided into three groups (I, II or III), based on their N-terminal sequences. In the present disclosure, any isoform of NRG-1 can be used. NRG isoforms can be generated from short transcripts leading directly to secreted ligands or are synthesized ligands or are synthesized as transmembrane precursor proteins.

NRG-1

In some embodiments, the NRG peptide is NRG-1β or a functional variant or fragment thereof. In more specific embodiments, the NRG-1 peptide or functional fragment thereof is NRG-1β isoform 1, isoform 2, isoform 3, isoform 4, isoform 5, isoform 6, isoform 7, isoform 8, isoform 9, isoform 10, isoform 11, or isoform 12. In particularly preferred embodiments, the isoform is GGF2.

In certain embodiments, the NRG-1β peptide or functional variant or fragment thereof is a recombinant protein. In another embodiment, the NRG-1β peptide or functional fragment thereof is a recombinant protein comprising the amino acid sequence of SEQ ID NO:19. In another embodiment, the NRG-1β peptide or functional fragment thereof is a recombinant protein comprising the amino acid sequence of SEQ ID NO:20.

Glial Growth Factor 2

In some embodiments the NRG peptide is glial growth factor, GGF2. The amino acid sequence of mature GGF2 is set forth in SEQ ID NO:1 and SEQ ID NO:2. In some embodiments, a peptide comprises a functional variant or fragment of GGF2. A functional fragment of GGF2 that binds to and activates an ErbB3 receptor on cardiac progenitor cells and activates ErbB signaling may comprise 371 amino acids or less, e.g., 370, 369, 368, 367, 366, 365, 360, 355, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 55, 50, 45, 40, 35, 30, 25, 20 amino acids, or less, of SEQ ID NO: 2.

A functional variant of GGF2 binds to and activates an ErbB3 receptor on cardiac progenitor cells and activates ErbB signaling. A variant GGF2 used in the methods of the invention may comprise an amino acid sequence selected from SEQ ID NO:3, SEQ ID NO:4, SEQ ID NO:5, SEQ ID NO:6, SEQ ID NO:7, SEQ ID NO:8, SEQ ID NO:9 or SEQ ID NO:10. In certain embodiments, the GGF2 peptide comprises an amino acid sequence that is 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:2 or functional fragment thereof.

NRG-2

In some embodiments the NRG peptide is an NRG-2 peptide, e.g., NRG-2α or NRG-2β. The amino acid sequence of NRG-2α is set forth in SEQ ID NO:21 and the amino acid sequence for NRG-2β is set forth in SEQ ID NO:2. In some embodiments, a peptide comprises a functional variant or fragment of NRG-2α or NRG-2β. A functional fragment of NRG-2α that binds to and activates an ErbB3 receptor on cardiac progenitor cells and activates ErbB signaling may comprise 329 amino acids or less, e.g., 325, 320, 310, 300, 290, 280, 270, 260, 250, 240, 230, 220, 210, 200, 190, 180, 170, 160, 150, 140, 130, 120, 110, 100, 90, 80, 70, 60, 55, 50, 45, 40, 35, 30, 25, 20 amino acids, or less, of SEQ ID NO:21. A functional fragment of NRG-2β that binds to and activates an ErbB3 receptor on cardiac progenitor cells and activates ErbB signaling may comprise 297 amino acids or less, e.g., 295, 290, 280, 270, 260, 250, 240, 230, 220, 210, 200, 190, 180, 170, 160, 150, 140, 130, 120, 110, 100, 90, 80, 70, 60, 55, 50, 45, 40, 35, 30, 25, 20 amino acids, or less, of SEQ ID NO:22.

A functional variant of NRG-2α or NRG-2β binds to and activates an ErbB3 receptor on cardiac progenitor cells and activates ErbB signaling. A variant NRG-2α or NRG-2β used in the methods of the invention may comprise an amino acid sequences that is 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:21 or SEQ ID NO:22, or a functional fragment thereof.

EGF-Like Domain

An NRG variant peptide or functional fragment thereof suitable for use in the methods of the disclosure comprises an EGF-like domain-containing peptide. In some embodiments the EGF-like domain-containing peptide comprises the amino acid sequences SEQ ID NO:11 or SEQ ID NO:12. In some embodiments, the NRG peptide or functional variant or fragment thereof used in the methods of the invention comprises an EGF-like domain derived from NRG-1β, particularly GGF2. In specific embodiments, the NRG peptide or functional variant or fragment thereof used in the methods of the invention comprises an EGF-like domain derived from GGF2. In other specific embodiments, the peptide or functional fragment thereof used in the methods of the disclosure comprises an EGF-like domain derived from NRG-1β, particularly GGF2. Exemplary EGF-like domain-containing peptides suitable for use in the methods of the invention, may comprise the amino acid sequence set forth in SEQ ID NO:13 (EGFL1), SEQ ID NO:14 (EGFL2), SEQ ID NO:15 (EGFL3), SEQ ID NO:16 (EGFL4), SEQ ID NO:17 (EGFL5), or SEQ ID NO:18 (EGFL6).

Compositions, Administration and Dosage

In certain embodiments, an NRG peptide or functional variant or fragment thereof suitable for use in the methods of the invention is a purified recombinant or chemically synthesized peptide.

In certain embodiments, an NRG peptide or functional variant or fragment thereof described herein, can be administered to subjects, e.g., humans, veterinary subjects, or experimental animals with a pharmaceutically-acceptable diluent, carrier, or excipient. Compositions of the disclosure can be provided in unit dosage form. Therapeutic formulations can be in the form of liquid solutions or suspensions; for oral administration, formulations can be in the form of tablets or capsules; and for intranasal formulations, in the form of powders, nasal drops, or aerosols.

Methods for making formulations are found in, for example, “Remington's Pharmaceutical Sciences.” Formulations for parenteral administration can, for example, contain excipients, sterile water, or saline, polyalkylene glycols such as polyethylene glycol, oils of vegetable origin, or hydrogenated napthalenes. Other potentially useful parenteral delivery systems for administering molecules of the disclosure include ethylene-vinyl acetate copolymer particles, osmotic pumps, implantable infusion systems, and liposomes. Formulations for inhalation can contain excipients, for example, lactose, or may be aqueous solutions containing, for example, polyoxyethylene-9-lauryl ether, glycocholate and deoxycholate, or can be oily solutions for administration in the form of nasal drops, or as a gel.

In certain embodiments, the NRG peptide or functional variant or fragment thereof provided herein is administered intermittently or discontinuously.

In accordance with the present disclosure, intermittent or discontinuous administration of a peptide described herein is directed to achieving a dosing regimen wherein narrow steady-state concentrations of the administered peptide are not maintained, thereby reducing the probability that the mammal will experience untoward side effects that may result from maintaining supraphysiological levels of the administered peptide over a prolonged duration. For example, side effects associated with supraphysiological levels of exogenously administered NRG include nerve sheath hyperplasia, mammary hyperplasia, renal nephropathy, hypospermia, hepatic enzyme elevation, heart valve changes, and skin changes at the injection site.

In a preferred embodiment, the present disclosure is directed to an intermittent dosing regimen that elicits or permits fluctuations in the serum levels of the NRG peptide or functional variant or fragment thereof, and thus reduces the potential for adverse side effects associated with more frequent administration of the peptide. The intermittent dosing regimen of the present disclosure thus confers therapeutic advantage to the mammal, but does not maintain steady state therapeutic levels of the peptide. As appreciated by those of ordinary skill in the art, there are various embodiments of the disclosure to obtain the intermittent dosing; the benefits of these embodiments can be stated in various ways for example, the administering does not maintain steady state therapeutic levels of the peptide, the administering reduces potential for adverse side effects associated with administration of a NRG peptide more frequently, and/or the like.

In certain embodiments, the NRG peptide or functional variant or fragment thereof, provides dosing intervals of at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof so long as the interval/regimen is at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer. In certain embodiments, the NRG peptide functional variant or fragment thereof, is administered at dosing intervals of at least once per month, once per 2 months, once per 3 months, or once per 6 months. For example, the peptide is administered on a dosing interval for at least 2 weeks, e.g., at least 2 weeks, 3 weeks, or 4 weeks. For example, the peptide is administered on a dosing interval of greater than 4 months.

In some embodiments, a therapeutically effective amount of the NRG peptide or a functional variant or fragment thereof, is administered to a mammal at dosing intervals of 48, 72, 96, or more hours. Preferably, a dosing regimen comprises administering a therapeutically effective amount of the peptide to a mammal at dosing intervals of 72, 96, or more hours. Accordingly, the present method calls for intermittent or discontinuous administration (every 72 to 96 hours, or even longer intervals) of the NRG peptide a functional variant or fragment thereof, to the mammal, wherein administration of the peptide is in an amount effective to treat, prevent, or delay progression of heart failure in the mammal. Dosing regimens for NRG, e.g., GGF2 or a functional fragment thereof, administration that do not maintain steady-state concentrations are equally as effective as more frequent dosing regimens, yet without the inconvenience, costs or side effects that can result from more frequent administration.

In certain embodiments, herein the term intermittent or discontinuous administration includes a regimen for dosing at least once every 2 weeks, once every 3 weeks, once every 4 weeks, once per month, once per 2 months, once per 3 months, once per 4 months, once per 5 months, once per 6 months, once per 7 months, once per 8 months, once per 9 months, once per 10 months, once per 11 months, or once per 12 months.

In certain embodiments of a dosing regimen described herein, the NRG peptide or functional variant or fragment thereof, is administered once every month, once every other month, once every three months, once every 3.5 months, once every 4 months, once every 4.5 months, once every 5 months, once every 6 months, once every 7 months, or on a less frequent dosing interval.

A dosing regimen of the disclosure can be initiated, established, or subsequently modified upon evaluation of a variety of factors, including, but not limited to ejection fraction, left ventricular ejection fraction, end-diastolic volume, end-systolic volume, heart volume, heart weight, liver toxicity, or increased or decreased protein expression levels in either cardiac tissue or blood samples of B-type Natiuretic Peptide, N-terminal B-type Natiuretic Peptide, and/or Troponin-I. A dosing regimen of the present disclosure can also be initiated, established, or subsequently modified upon evaluation of, amelioration of, or improvement of one or more symptoms of heart failure, e.g., shortness of breath, exercise intolerance, hospitalization, re-hospitalization, mortality, and/or morbidity. A change in one or more of these factors may indicate that the interval between doses may be too small, the administration too frequent, or the route of administration not optimal. In other cases, a change in one or more of these factors may indicate that an optimal dose and/or dosing interval has been reached, and optionally, may be maintained.

In some cases liver toxicity is monitored, such as at regular intervals, e.g., liver toxicity is assessed at least every 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

In some cases glucose levels, e.g., in plasma, serum, or blood of the subject, is monitored at regular intervals, e.g., liver toxicity is assessed at least every 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

For example, liver toxicity and/or glucose level is monitored on any dosing regimen described herein, e.g., on an escalating dosing regimen, a decreasing dosing regimen, and/or a dosing regimen in which a therapeutically effective dose is maintained and, e.g., not changed.

Conventional pharmaceutical practice is employed to provide suitable formulations or compositions, and to administer such compositions to subjects or animals. Any appropriate route of administration may be employed, for example, parenteral, intravenous, subcutaneous, intramuscular, transdermal, intracardiac, intraperitoneal, intranasal, aerosol, oral, or topical, e.g., by applying an adhesive patch carrying a formulation capable of crossing the dermis and entering the bloodstream, administration. For example, the route of administration is intravenous or subcutaneous injection/infusion. For example, the NRG peptide or functional variant or fragment thereof, may be administered by a route described herein, e.g., intravenous or subcutaneous injection/infusion. In other examples, the compositions are delivered via a catheter, a pump delivery system, or a stent.

Dose levels of the NRG peptide or functional variant or fragment thereof, for example, administered via injection, such as intravenous or subcutaneous injection, range from about 0.001 mg/kg to about 4 mg/kg bodyweight. For example, the doses levels of the peptide range from about 0.001 mg/kg to about 1.5 mg/kg, from about 0.007 mg/kg to about 1.5 mg/kg, from about 0.001 mg/kg to about 0.02 mg/kg, from about 0.02 mg/kg to about 0.06 mg/kg, from about 0.06 mg/kg to about 0.1 mg/kg, from about 0.1 mg/kg to about 0.3 mg/kg, about 0.02 mg/kg to about 0.75 mg/kg, from about 0.3 mg/kg to about 0.5 mg/kg, from about 0.5 mg/kg to about 0.7 mg/kg, from about 0.5 mg/kg to about 1.0 mg/kg, from about 0.7 mg/kg to about 1.0 mg/kg, from about 0.3 mg/kg to about 4 mg/kg, from about 0.3 mg/kg to about 3.5 mg/kg, from about 1.0 mg/kg to about 1.5 mg/kg, or from about 1 mg/kg to about 10 mg/kg.

In some cases, the dose levels of the NRG peptide or functional variant or fragment thereof, are equal to or less than about 1.5 mg/kg bodyweight, e.g., equal to or less than about 0.8 mg/kg, or less than about 0.756 mg/kg bodyweight.

For example, the dose levels of the NRG peptide or functional variant or fragment thereof, include about 0.007 mg/kg, about 0.02 mg/kg, about 0.06 mg/kg, about 0.19 mg/kg, about 0.38 mg/kg, about 0.76 mg/kg, or about 1.5 mg/kg bodyweight, e.g., 0.007 mg/kg, 0.021 mg/kg, 0.063 mg/kg 0.189 mg/kg, 0.378 mg/kg, 0.756 mg/kg, or 1.512 mg/kg bodyweight.

In some examples, the NRG peptide or functional variant or fragment thereof, is administered at a dose level of about 0.005 mg/kg to about 4 mg/kg bodyweight on a dosing interval of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

In other examples, the NRG peptide or functional variant or fragment thereof, is administered at a dose level of about 0.007 mg/kg, about 0.02 mg/kg, about 0.06 mg/kg, about 0.19 mg/kg, about 0.38 mg/kg, about 0.76 mg/kg, or about 1.5 mg/kg bodyweight on a dosing interval of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

In some cases, the NRG peptide or functional variant or fragment thereof, is administered at a dose level of 0.007 mg/kg, 0.021 mg/kg, 0.063 mg/kg, 0.189 mg/kg, 0.378 mg/kg, 0.756 mg/kg, or 1.512 mg/kg bodyweight on a dosing interval of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

In other cases, the NRG peptide or functional variant or fragment thereof, is administered at a dose level of about 0.35 mg/kg to about 3.5 mg/kg bodyweight, e.g., about 3.5 mg/kg, about 1.75 mg/kg, about 0.875 mg/kg, or about 0.35 mg/kg bodyweight, on a dosing interval of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

In some embodiments, the therapeutically effective amount of the NRG peptide or functional variant or fragment thereof, is about 0.06 mg/kg bodyweight to about 0.38 mg/kg bodyweight and the dosing interval is at least 2 weeks, e.g., at least 2 weeks, 3 weeks, 4 weeks, 1 month, 2 months, 3 months (quarterly), 4 months, 5 months, 6 months, 7 months, 8 months, 9 months, 10 months, 11 months, 12 months, or longer. For example, the therapeutically effective amount of a peptide described herein is about 0.063 mg/kg, about 0.189 mg/kg, or about 0.375 mg/kg. For example, a therapeutically effective amount of the peptide of about 0.063 mg/kg, about 0.189 mg/kg, or about 0.375 mg/kg is administered via intravenous injection or infusion, e.g., to prevent, treat, or delay the progression of heart failure.

In some cases, the NRG peptide or functional variant or fragment thereof, is administered at a dose level of about 0.056 mg/kg to about 0.57 mg/kg bodyweight, e.g., about 0.056 mg/kg, about 0.1 mg/kg, about 0.2 mg/kg, about 0.3 mg/kg, about 0.4 mg/kg, or about 0.57 mg/kg, on a dosing interval of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

The dose levels of the NRG peptide or functional variant or fragment thereof are administered via a route described above, e.g., intravenous or subcutaneous injection/infusion.

The dose level of the NRG peptide or functional variant or fragment thereof, when administered by a subcutaneous route may be equal to or greater than the dose level of the same peptide when administered by an intravenous route. Moreover, the length of intervals between doses may decrease or the frequency of dosing may increase when the peptide is administered by a subcutaneous route compared to an intravenous route. In certain embodiments, a subject who receives a peptide of the disclosure, by an intravenous route, and, subsequently demonstrates an increase of liver enzymes indicating liver toxicity, may be treated using an equivalent or greater dose of the peptide by a subcutaneous route.

Transdermal doses are generally selected to provide similar or lower blood levels than are achieved using injection doses.

In some dosing regimens of the present disclosure, an initial dose of a the NRG peptide or functional variant or fragment thereof, is administered to the subject, and subsequent doses (e.g., a second dose, a third dose, a fourth dose, and so on) are administered to the subject on a dosing interval described herein. In some cases, the initial dose is the same as one or more of the subsequent doses. For example, the initial dose is the same as all subsequent doses. In some cases, the initial dose is lower than one or more of the subsequent doses, e.g., as provided by an escalating dosing regimen described herein. In other cases, the initial dose is higher than one or more of the subsequent doses, e.g., as provided by a decreasing dosing regimen described herein.

Combination Treatment

The NRG peptide or functional variant or fragment thereof, can be administered as the sole active agent or they can be administered in combination with human cardiac progenitor cells or human embryonic stem cells. Additional agents may be administered with the NRG peptide or functional variant or fragment thereof, with or without human cardiac progenitor cells, including other compounds, e.g., peptides that demonstrate the same or a similar therapeutic activity and that are determined to be safe and efficacious for such combined administration. Other such compounds used for the treatment of CHF include brain natriuretic peptide (BNP); statins (e.g., atorvastatin, fluvastatin, lovastatin, pitavastatin, pravastatin, rosuvastatin, or simvastatin); drugs that block formation or action of specific neurohormones (e.g. angiotensin converting enzyme inhibitors (ACE-inhibitors), angiotensin receptor antagonists (ARBs), aldosterone antagonists and beta-adrenergic receptor blockers); inotropes (e.g. dobutamine, digoxin) to enhance cardiac contractility; vasodilators (e.g. nitrates, nesiritide); diuretics (e.g. furosemide) to reduce congestion; one or more antihypertensive agents (such as beta-blockers, ACE-inhibitors and ARBs); nitrates (e.g., isosorbide dinitrate); hydralazine; and/or calcium channel blockers.

In particular embodiments the NRG peptide or functional fragment or variant thereof, can be administered, with or without co-administration of human cardiac progenitor cells, to a subject in combination with azacitidine. In some embodiments azacitidine is administered concurrently with the NRG peptide as described herein, such as, e.g., a NRG-1β, particularly GGF2, NRG-2α, or NRG-2β, or functional variant or fragment thereof. In other embodiments, azacitidine is administered before, during or after administration with the NRG peptide or functional fragment thereof. In some embodiments, azacitidine promotes differentiation of cardiac progenitor cells. In more specific embodiments, azacitidine promotes differentiation of cardiac progenitor cells to myocytes.

In some embodiments, the NRG peptide or functional fragment or variant thereof, can be administered, with or without co-administration of human cardiac progenitor cells, in combination with benzodiazepine drug. The NRG peptide or functional variant or fragment thereof and the benzodiazepine drug may be administered to a subject within the same composition, or, alternatively, as part of the same treatment and/or in accordance with the same administration regimen as a peptide that comprises an EGF-like domain. Benzodiazepine drugs result from the fusion of a benzene ring and a diazepine ring. Benzodiazepine drugs may be classified as short-, intermediate-, or long-acting. Benzodiazepine drugs share anxiolytic, sedative, hypnotic, muscle relaxant, amnesic, anticonvulsant, and anti-hypertension properties. Exemplary benzodiazepine drugs of the disclosure include, but are not limited to, alprazolam, bretazenil, bromazepam, brotizolam, chlorodiazepoxide, cinolazepam, clobazam, clonazepam, clorazepate, clotiazepam, cloxazolam, delorazepam, diazepam, estazolam, eszopicloneetizolam, ethyl loflazepate, flumazenil, flunitrazepam, 5-(2-bromophenyl)-7-fluoro-1H-benzo[e][1,4]diazepin-2(3H)-one, flurazepam, flutoprazepam, halazepam, ketazolam, loprazolam, lorazepam, lormetazepam, medazepam, midazolam, nimetazepam, nitrazepam, nordazepam, oxazepam, phenazepam, pinazepam, prazepam, premazepam, purazolam, quazepam, temazepam, tetrazepam, triazolam, zaleplon, zolpidem, and zopiclone. The following exemplary benzodiazepine drugs may have anxiolytic properties: alprazolam, bretazenil, bromazepam, chlorodiazepoxide, clobazam, clonazepam, clorazepate, clotiazepam, cloxazolam, delorazepam, diazepam, etizolam, ethyl loflazepate, halazepam, ketazolam, lorazepam, medazepam, nordazepam, oxazepam, phenazepam, pinazepam, prazepam, premazepam, and purazolam. The following exemplary benzodiazepine drugs may have anticonvulsant properties: bretazenil, clonazepam, clorazepate, cloxazolam, diazepam, flutoprazepam, lorazepam, midazolam, nitrazepam, and phenazepam. The following exemplary benzodiazepine drugs may have hypnotic properties: brotizolam, estazolam, eszopiclone, flunitrazepam, flurazepam, flutoprazepam, loprazolam, lormetazepam, midazolam, nimetazepam, nitrazepam, quazepam, temazepam, triazolam, zaleplon, zolpidem, and zopiclone. The following exemplary benzodiazepine drug may have sedative properties: cinolazepam. The following exemplary benzodiazepine drugs may have muscle relaxant properties: diazepam and tetrazepam.

In particular embodiments the NRG peptide or functional fragment or variant thereof, can be administered, with or without co-administration of human cardiac progenitor cells, in combination with midazolam to a subject. Midazolam may be administered with the NRG peptide or functional variant or fragment within the same composition, or, alternatively, as part of the same treatment and/or in accordance with the same administration regimen as the NRG peptide or functional variant or fragment thereof. Although a benzodiazepine drug, e.g. midazolam, may be administered according to any dosing regimen described in the disclosure, in particular embodiments, the benzodiazepine drug, e.g. midazolam, may be administered in one or more doses, including oral doses. In certain embodiments, when the benzodiazepine drug, e.g. midazolam, is administered in one or more doses, including oral doses, the NRG peptide or functional variant or fragment thereof is administered in a single dose, e.g. a single intravenous infusion. The benzodiazepine drug, e.g. midazolam, may be administered prior to, simultaneously with, or following a dose of the NRG peptide or functional variant or fragment thereof. In a particular embodiment, a benzodiazepine drug, e.g. midazolam, is administered in 5 oral doses, after the second of which, the NRG or functional variant or fragment thereof, is administered in a single dose, e.g. a single intravenous infusion.

Utility of the Methods

In conjunction with cardiac catheterization or heart surgery, it can be determined whether a subject harbors a population of cardiac progenitor cells that is responsive to NRG. Typically, this analysis would be conducted by taking a biopsy from the subject's heart tissue and screening the tissue for markers of cardiac progenitor cells, such as, e.g., Sca-1 and c-Kit. The presence of ErbB2 and ErbB3 receptors will also indicate the presence of cardiac progenitor cells. Once the subject has been identified as having an appropriate population of cardiac progenitor cells, the cells are exposed to an NRG peptide or functional variant or fragment thereof to determine whether the cells respond by exhibiting reduced conversion to fibroblasts and myofibroblasts and/or by preferentially differentiating into cardiac myocytes. Subjects whose cardiac progenitor cells demonstrate this response to the NRG peptide or functional variant or fragment thereof are then treated with an NRG peptide or functional fragment or variant thereof. These methods can be used in conjunction with any present cardiac injury, suspected cardiac injury, or anticipated cardiac injury, including heart failure.

Heart failure in humans begins with reduced myocardial contractility, which leads to reduced cardiac output. The methods provided herein can be used to augment heart function, reduce scar tissue, and regenerate healthy heart tissue. For example, the methods described herein can be used, following a determination that a subject harbors cardiac progenitor cells that are responsive to NRG, to promote differentiation of cardiac progenitor cells toward cardiac myocytes and suppress the conversion of cardiac progenitor cells into fibroblasts and myofibroblasts cells in an area of the heart that has been damaged or become ischemic.

In one aspect, the methods as disclosed herein can be used to regenerate cardiac tissue, repair cardiac tissue or decrease cardiac fibrosis after cardiac injury. In another aspect, the methods described herein prevent the onset of cardiac injury.

Suitable subjects or subjects include mammals. Mammals include, but are not limited to, humans, mice, rats, rabbits, dogs, monkeys or pigs. In one embodiment of the disclosure, the mammal is a human.

In certain embodiments cardiac injury results from a cardiovascular disease. One of skill in the art would appreciate the numerous cardiovascular diseases. In specific embodiments, the cardiovascular disease can result from; e.g., coronary artery disease; heart failure; stroke; myocardial infarction; cardiomyopathy; hypertension; ischemic heart disease; atrial fibrillation; congenital heart disease; myocarditis; endocarditis; periocarditis; atherosclerosis; vascular disease; left ventricular systolic dysfunction; coronary bypass surgery; exposure to a cardiotoxic compound; thyroid disease; viral infection; gingivitis; drug abuse; alcohol abuse, or high blood cholesterol.

In some embodiments, subjects of the methods provided in this disclosure may present with chronic heart failure. In a preferred embodiment, the subject's condition has remained stable for at least 1, 2, 3, 4, 5, or 6 months. Stable or chronic heart failure may be further characterized by the lack of increase or decrease in heart function and/or damage over a period of at least 1, 2, 3, 4, 5, or 6 months. For example, the subject has suffered from chronic heart failure for at least 1 month, e.g., at least 1, 2, 3, 4, 5, 6, or more months, prior to administration of a peptide as described herein.

For example, the subject suffers from class 2, 3, or 4 heart failure prior to administration of a peptide as described herein. The New York Heart Association Functional Classification system is used to determine the class of heart failure based on how much the subject is limited during physical activity. Subjects who fall under class 1 heart failure have cardiac disease but no limitation of physical activity. Ordinary physical activity does not cause excessive fatigue, palpitation, dyspnea or anginal pain. Subjects who fall under class 2 heart failure have cardiac disease that results in slight limitation of physical activity. These subjects are comfortable at rest, but ordinary physical activity causes fatigue, palpitation, dyspnea or anginal pain. Class 3 heart failure subjects have cardiac disease that results in significant limitation of physical activity. Although these subjects are comfortable at rest, less than ordinary physical activity results in fatigue, palpitation, dyspnea or anginal pain. Class IV heart failure subjects have cardiac disease that results in an inability to perform any physical activity without discomfort. At rest, these subjects may experience symptoms of heart failure or anginal syndrome. Any physical activity increases the discomfort level.

In some embodiments, the subject suffers from systolic heart failure. For example, the subject suffers from systolic left ventricular dysfunction. For example, the subject has a left ventricular ejection fraction of 40% or less, e.g., 40%, 35%, 30%, 25%, 20%, 15%, 10%, or less, prior to administration of peptide described herein.

In some examples, the subject is a human of at least 18 years of age, e.g., at least 18, 20, 25, 30, 35, 40, 45, 50, 55, 60, 65, 70, 75, 80, 85, 90, or 95. In some cases, the human is between 18-75 years of age. In some examples, the subject is a human between 13-18 years old.

In some cases, the subject may suffer from acute decompensated heart failure (ADHD) prior to administration of a peptide described herein. For example, acute decompensated heart failure is characterized by a sudden or gradual onset of one or more symptoms or signs of heart failure that requires emergency room visits, hospitalization, and/or unplanned doctor office visits. In some cases, ADHD is associated with pulmonary and/or systemic congestion, which may be caused by an increase in left and/or right heart filling pressures. See, e.g., Joseph et al., 2009. For example, ADHD can be diagnosed by measuring the level of plasma B-type natriuretic peptide (BNP) or N-terminal pro-B-type natriuretic peptide (NT-proBNP) in a subject, using methods commonly known in the art. For example, a BNP level in a biological sample (such as blood, plasma, serum, or urine) from a subject that is higher than 100 pg/dL, e.g., at least 100 pg/dL, 200 pg/dL, 300 pg/dL, 400 pg/dL, 500 pg/dL, 600 pg/dL or higher, may indicate that a subject has ADHD. In some examples, a therapeutic dosing regimen of a peptide described herein is sufficient to prevent, reduce, or delay the occurrence of ADHD.

In some embodiments, the heart failure may result from hypertension, ischemic heart disease, exposure to a cardiotoxic compound, e.g., cocaine, alcohol, an anti-ErbB2 antibody or anti-HER antibody, such as HERCEPTIN®, or an anthracycline antibiotic, such as doxorubicin or daunomycin, myocarditis, thyroid disease, viral infection, gingivitis, drug abuse, alcohol abuse, pericarditis, atherosclerosis, vascular disease, hypertrophic cardiomyopathy, acute myocardial infarction or previous myocardial infarction, left ventricular systolic dysfunction, coronary bypass surgery, starvation, radiation exposure, an eating disorder, or a genetic defect.

In another embodiment of the disclosure, an anti-ErbB2 or anti-HER2 antibody, such as HERCEPTIN®, is administered to the mammal before, during, or after anthracycline administration.

In other embodiments of the disclosure, a subject's cardiac progenitor cells are tested for responsiveness to NRG and if responsive, the NRG peptide or functional variant or fragment thereof, is administered prior to exposure to a cardiotoxic compound, during exposure to the cardiotoxic compound, or after exposure to the cardiotoxic compound; the NRG peptide or functional variant or fragment thereof is administered prior to or after the diagnosis of congestive heart failure in the mammal. A method of the disclosure can take place after the subject mammal has undergone compensatory cardiac hypertrophy. In some examples, an outcome of a method described herein is to maintain left ventricular hypertrophy, to prevent/delay progression of myocardial thinning, or to inhibit cardiomyocyte apoptosis. In other embodiments of the disclosure, the NRG peptide or functional variant or fragment thereof is administered either prior to or after the diagnosis of congestive heart failure in the mammal. In yet another embodiment of the disclosure, the NRG peptide or functional variant or fragment thereof is administered to a mammal that has undergone compensatory cardiac hypertrophy. In other particular embodiments of the disclosure, administration of the NRG peptide or functional variant or fragment thereof maintains left ventricular hypertrophy, prevents/delays progression of myocardial thinning, and/or inhibits cardiomyocyte apoptosis.

In other embodiments, a subject in need of testing for cardiac progenitor cells that are responsive to neuregulin and a treatment or prophylaxis described herein is at risk for heart failure, e.g., congestive heart failure. Risk factors that increase the likelihood of an individual's developing heart failure are well known. These include, and are not limited to, smoking, obesity, high blood pressure, ischemic heart disease, vascular disease, coronary bypass surgery, myocardial infarction, left ventricular systolic dysfunction, exposure to cardiotoxic compounds (alcohol, drugs such as cocaine, and anthracycline antibiotics such as doxorubicin and daunorubicin), viral infection, pericarditis, myocarditis, gingivitis, thyroid disease, radiation exposure, genetic defects known to increase the risk of heart failure (such as those described in Bachinski and Roberts, 1998; Siu et al., 1999; and Arbustini et al., 1998), starvation, eating disorders such as anorexia and bulimia, family history of heart failure, and myocardial hypertrophy. This risk may be reduced by determining whether the subject has cardiac progenitor cells that are responsive to NRG and then administering the NRG peptide or functional variant or fragment thereof as described herein if the cardiac progenitor cells are determined to be responsive. Alternatively, the risk may be reduced by administering a population of cardiac progenitor cells to the subject found to have cardiac progenitor cells that are responsive to NRG and simultaneously or sequentially administering the NRG peptide or functional variant or fragment thereof described herein.

In accordance with the present disclosure, the NRG peptide or functional variant or fragment thereof can be administered intermittently to a subject found to have cardiac progenitor cells that are responsive to NRG to achieve prophylaxis such as by preventing or delaying/decreasing the rate of congestive heart disease progression in those identified as being at risk. For example, administration of the peptide to a subject in early compensatory hypertrophy permits maintenance of the hypertrophic state and prevents/delays the progression to heart failure. In addition, those identified to be at risk may be given cardioprotective treatment with the peptide prior to the development of compensatory hypertrophy if the subject is found to have cardiac progenitor cells that are responsive to NRG.

Administration of the NRG peptide or functional variant or fragment thereof described herein to cancer subjects found to have cardiac progenitor cells that are responsive to NRG prior to and during anthracycline chemotherapy or anthracycline/anti-ErbB2 (anti-HER2) antibody, e.g., HERCEPTIN®, combination therapy can prevent/delay a subject's cardiomyocytes from undergoing apoptosis, thereby preserving cardiac function. Subjects who have already suffered cardiomyocyte loss also derive benefit from NRG treatment, because the remaining myocardial tissue responds to NRG exposure by displaying hypertrophic growth and increased contractility.

Exemplary metrics of heart function include but are not limited to ventricular ejection fraction (EF), e.g., left ventricular ejection fraction (LVEF), end systolic volume (ESV), end diastolic volume (EDV), fractional shortening (FS), number of hospitalizations, exercise tolerance, mitral valve regurgitation, dyspnea, peripheral edema, and occurrence of ADHD. An improvement in heart function, e.g., as a result of administration of a peptide as disclosed herein, is detected, e.g., by one or more of the following: an increase in LVEF, a decrease in ESV, a decrease in EDV, an increase in FS, a decrease in the number of hospitalizations, an increase in exercise tolerance, a decrease in the number of occurrences in or the severity of mitral valve regurgitation, a decrease in dyspnea, a decrease in peripheral edema, and prevention or reduction in occurrence of ADHD. In some examples, where a subject suffers from heart failure with preserved LVEF, a metric of heart function includes but is not limited to ESV, EDV, FS, number of hospitalizations, exercise tolerance, mitral valve regurgitation, dyspnea, occurrence of ADHD, and peripheral edema.

In some examples, administration of a therapeutically effective amount of the NRG peptide or functional variant or fragment thereof to a subject found to have cardiac progenitor cells that are responsive to NRG, or a subject who has received cardiac progenitor cells that are responsive to NRG, is sufficient to increase the LVEF in the subject by at least 1%, e.g., at least 1, 2, 3, 4, 5, 6, 7, 8, 9, 10, 15, 20, 25, 30%, or greater, compared to the LVEF prior to administration of the peptide. For example, the increase in LVEF is at least 1-20%. In some cases a therapeutically effective amount of the NRG peptide or functional variant or fragment thereof as described herein is sufficient to increase the LVEF of the subject in need thereof to an ejection fraction of about 10-40%, e.g., the LVEF of the subject is increased to an ejection fraction of about 10%, 15%, 20%, 25%, 30%, 35%, or about 40%. In other cases, a therapeutically effective amount of the NRG peptide or functional variant or fragment thereof is sufficient to increase the LVEF of the subject in need thereof to an ejection fraction of about 40-60%, e.g., the LVEF of the subject is increased to an ejection fraction of about 40%, 45%, 50%, 55%, or about 60%. In yet other cases a therapeutically effective amount of the NRG peptide or functional variant or fragment thereof is sufficient to completely restore the LVEF of the subject in need thereof to a normal LVEF value. For example, the LVEF of the subject increases within 90 days or less, e.g., within 90 d, 80 d, 70 d, 60 d, 50 d, 40 d, 30 d, 20 d, 10 d, or less, of the first administration, e.g., initial dose, of the NRG peptide or functional variant or fragment thereof in the subject. In some cases, the increased LVEF in the subject is maintained for at least 12 hours, e.g., at least 12 hours, 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, following the first administration of the peptide, e.g., without a subsequent administration of the peptide. For example, a therapeutically effective dose of a peptide described herein is sufficient to maintain and/or stabilize the LVEF in the subject for at least 12 hours, e.g., at least 12 hours, 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, following the first administration of the peptide, e.g., without a subsequent administration of the peptide.

In some examples, administration of a therapeutically effective amount of the NRG peptide or functional variant or fragment thereof is sufficient to decrease the EDV in the subject by at least 1 mL, e.g., at least 1 mL, 5 mL, 10 mL, 15 mL, 20 mL, 25 mL, 30 mL, 40 mL, 50 mL, 60 mL, 70 mL, 80 mL, 90 mL, 100 mL, or greater, e.g., at least 1-60 mL, compared to the EDV of the subject prior to administration of the peptide. For example, the EDV of the subject decreases within 90 days or less, e.g., within 90 d, 80 d, 70 d, 60 d, 50 d, 40 d, 30 d, 20 d, 10 d, or less, of the first administration of the peptide in the subject, e.g., the initial dose of the peptide. In some cases, the decreased EDV in the subject is maintained for at least 12 hours, e.g., at least 12 hours, 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, following the first administration of the peptide, e.g., without a subsequent administration of the peptide.

In other examples, administration of a therapeutically effective amount of the NRG peptide or functional variant or fragment thereof to a subject found to have cardiac progenitor cells that are responsive to NRG is sufficient to decrease the ESV in the subject by at least 1 mL, e.g., at least 1 mL, 5 mL, 15 mL, 20 mL, 25 mL, 30 mL, 40 mL, 50 mL, 60 mL, 70 mL, 80 mL, 90 mL, 100 mL, or greater, e.g., at least 1-30 mL, compared to the ESV of the subject prior to administration of the peptide. For example, the ESV of the subject decreases within 90 days or less, e.g., within 90 d, 80 d, 70 d, 60 d, 50 d, 40 d, 30 d, 20 d, 10 d, or less, of the first administration of the peptide in the subject, e.g., the initial dose of the peptide. In some cases, the decreased ESV in the subject is maintained for at least 12 hours, e.g., at least 12 hours, 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, following the first administration of the peptide, e.g., without a subsequent administration of the peptide.

In some cases, administration of a therapeutically effective amount of the NRG peptide or functional variant or fragment thereof to a subject found to have cardiac progenitor cells that are responsive to NRG is sufficient to increase the FS in the subject by at least 1%, e.g., at least 1, 2, 3, 4, 5, 6, 7, 8, 9, 10, 15, 20, 25, 30%, or greater, compared to the FS prior to administration of the peptide. For example, the increase in FS is at least 1-15%. In some cases a therapeutically effective amount of a peptide described herein is sufficient to increase the FS of the subject in need thereof to a Percent Fractional Shortening of about 15%, e.g. about 1%, 2%, 3%, 4%, 6%, 7%, 8%, 9%, 10%, or about 15%. In other cases a therapeutically effective amount of a peptide described herein is sufficient to increase the FS of the subject in need thereof to a Percent Fractional Shortening of about 15-20%, e.g., about 15%, 16%, 17%, 18%, 19%, or about 20%. In yet other cases a therapeutically effective amount of a peptide described herein is sufficient to increase the FS of the subject in need thereof to a Percent Fractional Shortening of about 20-25%, e.g., about 20%, 21%, 22%, 23%, 24%, or about 25%. In further cases, a therapeutically effective amount of a peptide described herein is sufficient to increase the FS of the subject in need thereof to a Percent Fractional Shortening of about 25-45%, e.g., about 25%, 26%, 27%, 28%, 29%, 30%, 31%, 32%, 33%, 34%, 35%, 36%, 37%, 38%, 39%, 40%, 41%, 42%, 43%, 44%, or about 45%. For example, the FS of the subject increases within 90 days or less, e.g., within 90 d, 80 d, 70 d, 60 d, 50 d, 40 d, 30 d, 20 d, 10 d, or less, of the first administration of the peptide in the subject, e.g., the initial dose of the peptide. In some cases, the increased FS in the subject is maintained for at least 12 hours, e.g., at least 12 hours, 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, following the first administration of the peptide, e.g., without a subsequent administration of the peptide.

The metrics for assessing heart function described herein are determined by methods commonly known in the art.

### REFERENCES

1. Arbustini et al., Heart 80:548-558, 1998
2. Bachinski and Roberts, Cardiol. Clin. 16:603-610, 1998
3. Carraway et al., Nature 387:512-516, 1997
4. Chang et al., Nature 387:509-512, 1997
5. Cohn et al., N. Engl. J Med. 339:1810-1816, 1998
6. Galindo et al., Journal of American Heart Association 3:e000773, 2014
7. Falls. Exp. Cell Res. 284(1):14-30, 2003.
8. Higashiyama et al., J. Biochem. 122:675-680, 1997
9. Hijazi et al., Int. J. Oncol. 13:1061-1067, 1998
10. Holmes et al., Science 256:1205-1210, 1992
11. Joseph et al., Tex. Heart Inst. J. 36(6):510-20, 2009
12. Lemke, Mol. Cell. Neurosci. 7:247-262, 1996
13. Lenihan et al., American College of Chemistry Meeting, Mar. 9-11, 2013
14. No authors listed, Am. J. Cardiol., 83(2A):1A-38-A, 1999
15. Peles and Yarden, BioEssays 15:815-824, 1993
16. Sawyer and Caggiano, J. Mol. Cell Cardiol. 51(4):501-5, 2011
17. Siu et al., Circulation 8:1022-1026, 1999
18. SOLVD Investigators, N. Engl. J. Med. 327:685-691, 1992

The embodiments described herein are intended to be merely exemplary, and those skilled in the art will recognize, or be able to ascertain using no more than routine experimentation, numerous equivalents to the specific procedures described herein. All such equivalents are considered to be within the scope of the present disclosure and are covered by the following embodiments. All references (including patent applications, patents, and publications) cited herein are incorporated herein by reference in their entireties and for all purposes to the same extent as if each individual publication or patent or patent application was specifically and individually indicated to be incorporated by reference in its entirety for all purposes.

### Example 1. General Methods

Isolation of Cardiac Cells

Isolation of murine cardiac cells was performed after digestion of heart ventricles using 10 mg/ml collagenase II, 2.5 U/ml dispase II, 1 μg/ml DNase I, and 2.5 mM CaCl₂ for 20 min at 37° C. Cardiac CD31^{pos}CD45^{neg} endothelial cells and Sca-1^{pos}CD31^{neg} progenitor cells were isolated using magnetic-activated cell-sorting MicroBead technology (Miltenyi Biotec, Inc).

Single-cell clonogenic techniques were used to isolate human-cardiac progenitor cells. Human cardiac progenitor cells were isolated from anterior left ventricle free wall epicardial biopsies obtained from subjects undergoing bypass surgery. Single cell suspension was prepared using collagenase II/Dispase II/CaCL2 digestion solution and plated in 48 culture dish at the concentration of 10³ cells per cm². Hematopoietic cells were removed by magnetic separation using human CD45 microbeads. CD45-depleted cells were plated on 48-well plates at a density of 10³ cell per cm² in M199-EGM-2 medium. The wells were analyzed for growing colonies twice weekly. Rapidly growing clones (2-3 colonies/sample) were harvested, resuspended in fresh growth medium, and plated on 0.1% gelatin-coated tissue culture dishes at a density of 5×10³ cells/cm². Human cardiac progenitor cells were characterized by cell surface expression of CD105 and absence of CD31 endothelial marker, CD45, and CD117/c-Kit hematopoetic markers.

Induction of Cardiomyogenic Differentiation

To induce cardiomyogenic differentiation, cardiac progenitor cells were pretreated with 5 μM 5′-azacytidine for 72 h in DMEM-LG medium supplemented with 10% FBS and cultured in DMEM-LG medium supplemented with 2% FBS, 1 ng/ml TGFβ, 100 μM ascorbic acid, 0.2% DMSO, and 10 ng/ml bFGF changed every 3 days.

Differentiation of cardiac progenitor cells towards endothelial cells was induced by incubation of cells in 15% FBS 199 medium containing 20 ng/ml of VEGF.

Induction of Myocardial Infarction

Myocardial infarction in mice was induced by permanent ligation of the left coronary artery.

Any known technique to the skilled artisan can be used to induce myocardial infarction. For example, myocardial infarction can be induced by angiographically guided intracoronary balloon occlusion (see, e.g., Galindo et al., 2014).

Immunohistochemistry

Intracellular staining for αSMA and collagen type I was performed in fixed and permeabilized cells (Cytofix/Cytoperm kit, BD Biosciences) using monoclonal FITC-conjugated anti-αSMA (Sigma) and biotin-conjugated anti-collagen type I (600-401-103; Rockland, Inc., Rockland, Pa.) antibodies. Mouse IgG2a-FITC- (Sigma) and biotin-conjugated rabbit whole IgG (Jackson ImmunoResearch, Inc., West Grove, Pa.) were used as an isotype control. Viable and non-viable cells were distinguished using LIVE/DEAD Fixable Stain kit (Life Technologies, Carlsbad, Calif.).

Flow cytometric analysis was performed using a LSRII flow cytometer and the data were analyzed with WinList 5.0 software.

Recombinant NRG Proteins

Following established approaches as described in International Publication Nos. WO2010030317 and WO2014138502, multiple NRG splice variants were cloned and produced.

### Example 2. Expression of NRG Receptors in the Heart

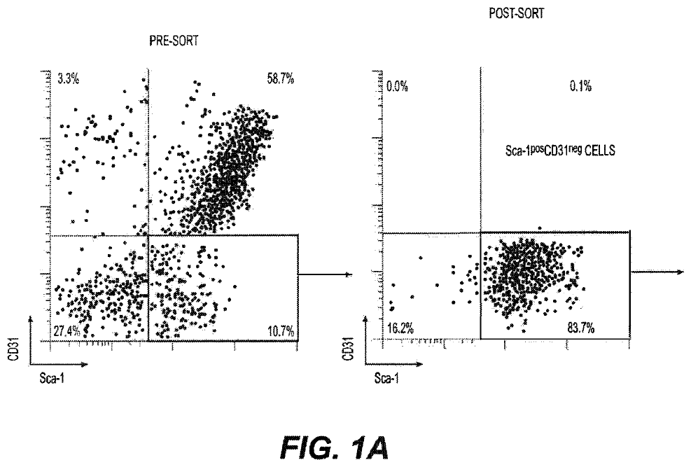

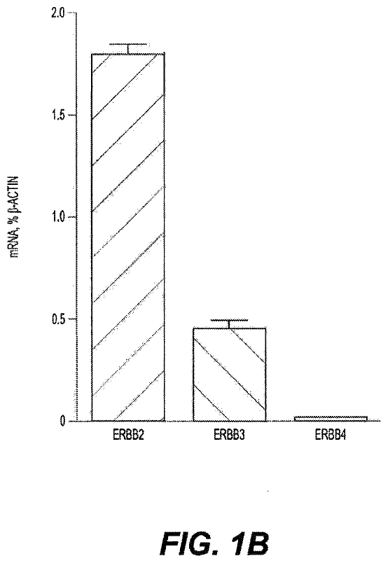

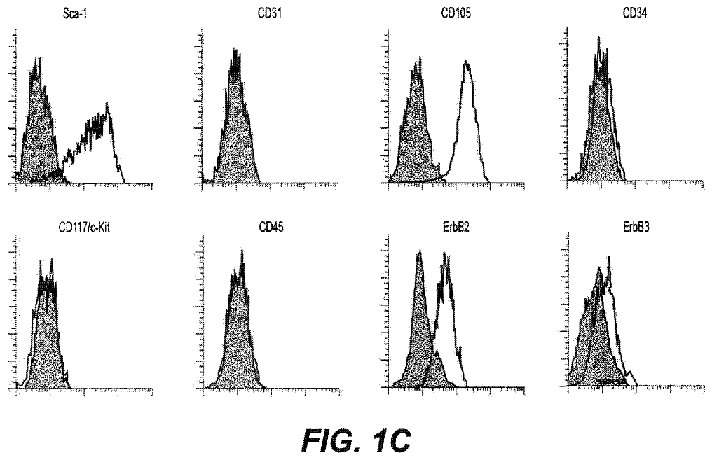

To determine the purity of isolated subpopulations of cardiac progenitor cells, pre- and post-magnetic-activated cell sorting was performed (FIG. 1A). Next, in order to determine which of the NRG receptors (i.e., ErbB2, ErbB3, Erb4) were expressed in murine cardiac progenitor cells, mRNA expression of ErbB receptors was analyzed. As shown in FIG. 1B, mouse cardiac Sca-1^{pos}CD31^{neg} stromal cells expressed functional ErbB2 and ErbB3 receptors. The expression of NRG receptors was confirmed by analyzing flow cytometry histograms of cell surface markers on Sca-1^{pos}/CD31^{neg} cardiac progenitor cells (FIG. 1C). These studies showed that murine cardiac progenitor cells expressed high levels of ErbB2, moderate levels of Erb3, and very little ErbB4 (FIG. 1B).

### Example 3. Differentiation of Murine Cardiac Progenitor Cells Towards Endothelial Cells and Cardiac Myocytes In Vitro

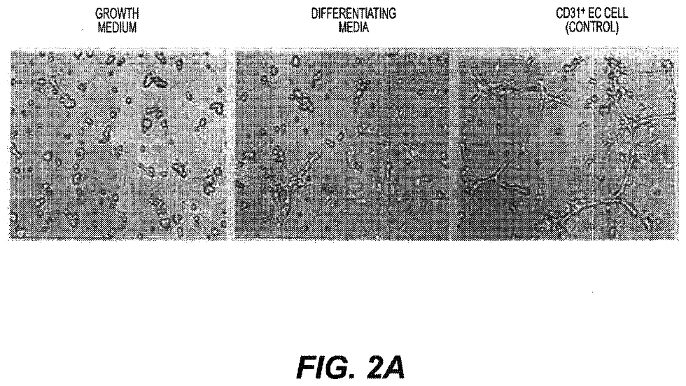

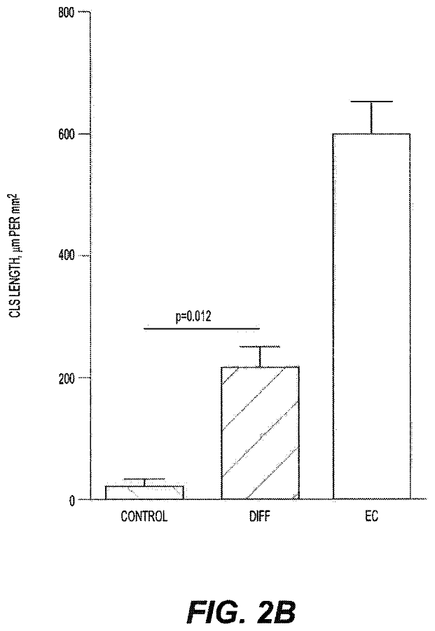

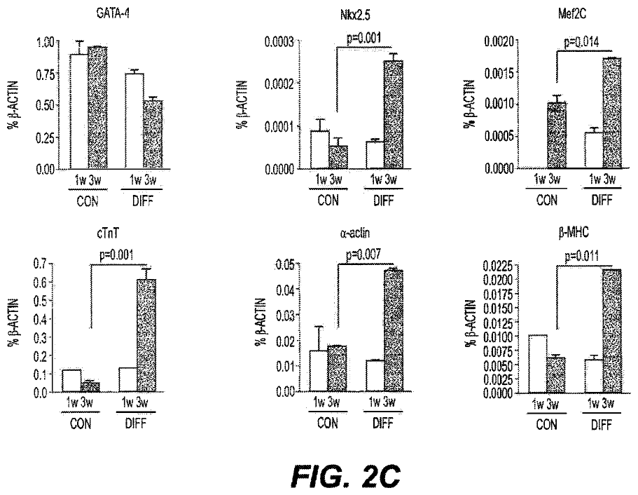

FIG. 2A shows micrographs of capillary-like structure formation after incubation of cardiac progenitor cells in growth medium or endothelial cell-differentiating media. CD31^{pos} cardiac endothelial cells were used as a positive control. FIG. 2B shows graphical representation of morphogenic activity of cardiac progenitor cells, incubated in growth medium or in endothelial cells differentiating media, and cardiac endothelial cells. Capillary tube formation was estimated by measuring their total length. Cardiac-specific gene expression in cardiac Sca-1^{pos}CD31^{neg} cells cultured in normal growth medium or in cardiac myocyte-differentiating media for 1 or 3 weeks was analyzed using real-time RT-PCR (FIG. 2C). This study shows that murine cardiac progenitor cells differentiate towards endothelial cells and cardiac myocytes. Values are averages of three experiments.

### Example 4. NRG-1 Prevents Transition of Murine Cardiac Progenitor Cells into Myofibroblasts

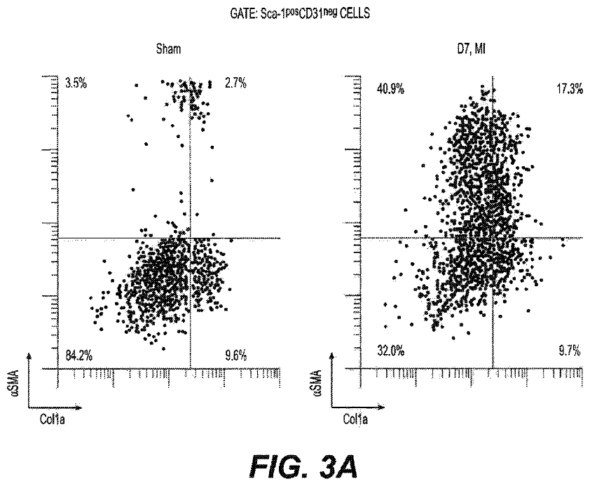

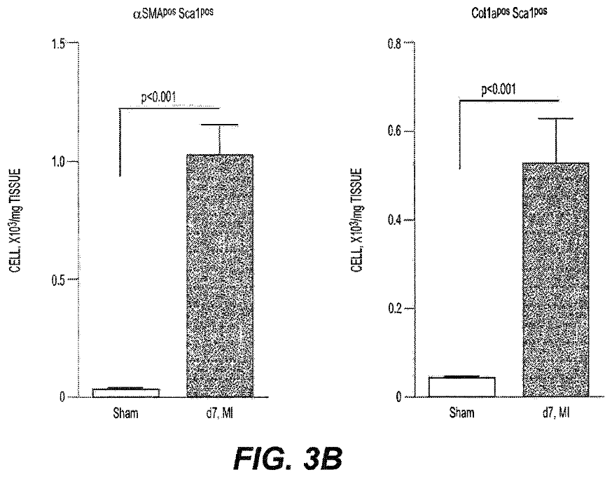

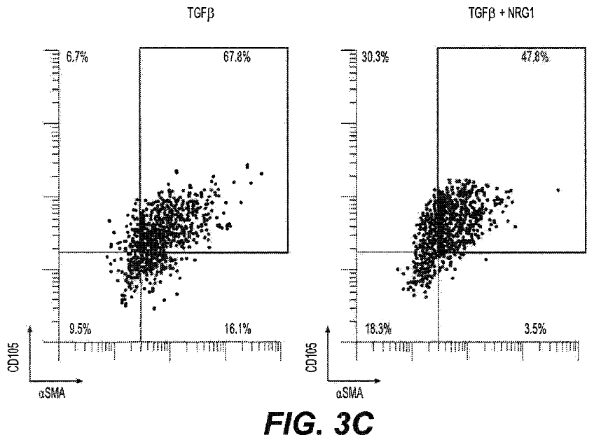

Representative flow cytometric dot plots demonstrate that cardiac progenitor cells accumulate towards αSMA positive and collagen 1 producing myofibroblasts in vivo on day 7 after experimental myocardial infarction in mice (FIG. 3A). FIG. 3B shows graphical representative data from flow cytometric analysis of αSMA positive and collagen 1α positive Sca-1^{pos}CD31^{neg} cardiac progenitor cells. P values are indicated, unpaired t test. The expression of αSMA protein in cardiac progenitor cells incubated with TGFβ alone or in combination with 30 ng/ml NRG-1 for 48 hrs. is illustrated in representative cytofluorographic dot plots in FIG. 3C. Interestingly, this experiment shows that NRG-1 reduces myofibroblast expression. In addition, the mean fluorescence intensity of αSMA expression in cardiac Sca-1^{pos}CD31^{neg} progenitor cells was assessed by flow cytometry (FIG. 3D). Data represent mean±SEM from three independent experiments. P-values indicate significance level calculated by t-test.

### Example 5. Localization of Erb2 and ErbB3 Receptors in Vascular and Peri-Vascular Regions in the Human Heart

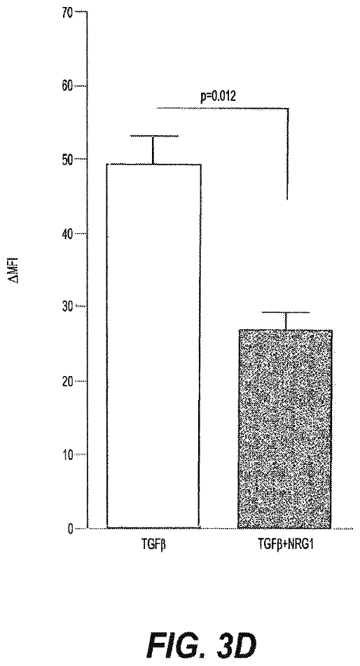

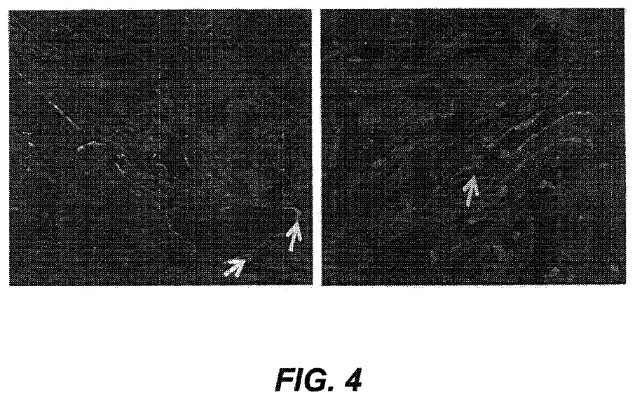

This study examines the localization of NRG receptors in the human heart. FIG. 4 shows that a similar expression of ErbB receptors in murine hearts exists in cardiac^{pos}CD31^{neg} progenitor cells derived from the human heart. Specifically, ErbB2 and ErbB3 receptors are localized in vascular/peri-vascular regions of the human heart (FIG. 4)

### Example 6. NRG-1 Prevents Transition of Human Cardiac Progenitor Cells into Myofibroblasts

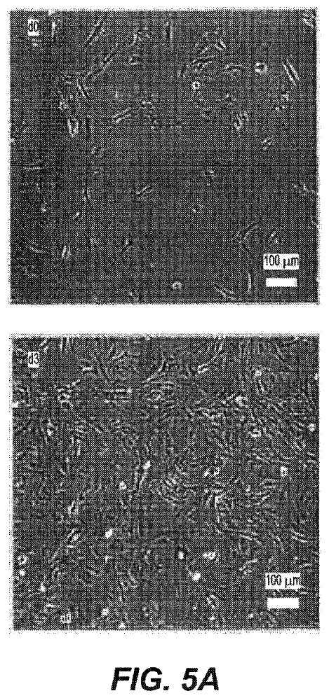

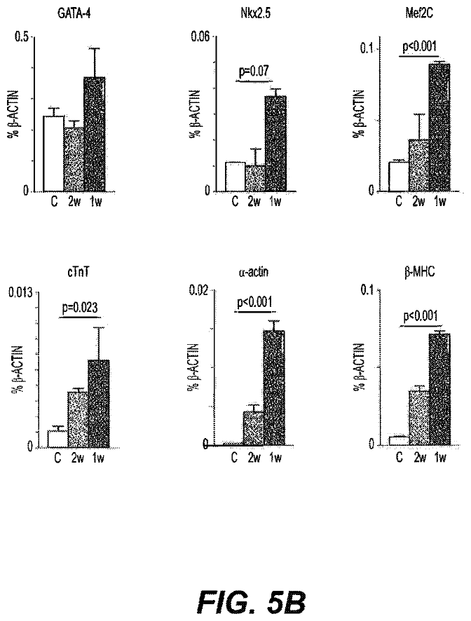

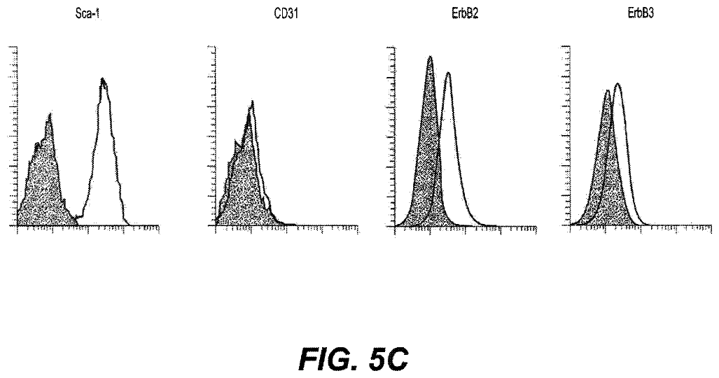

This study investigated the effects of stimulating cardiac progenitor cells with NRG-1. FIG. 5A shows exemplary phase contrast micrographs of human cardiac progenitor cells immediately after replating a single cell-derived clone and 3 days later. Scale bar=100 μm. Next, cardiac-specific gene expression in human cardiac progenitor cells before and after culturing in differentiating media for 1 or 2 weeks was analyzed using real-time PCR (FIG. 5B). Values are averages of three experiments, unpaired t test. FIG. 5C shows representative flow cytometry histograms of cell surface markers on human cardiac progenitor cells. Shaded areas represent the fluorescence of cells treated with corresponding isotype-matching antibody controls. These studies showed that stimulation of cells with NRG-1 and endogenous ErbB receptor ligand prevents their transition into myofibroblasts.

1-49. (canceled)

50. A method of treating or preventing cardiac injury in a subject, comprising:
a) exposing one or more cardiac progenitor cells from the subject to a neuregulin peptide or a functional variant or fragment thereof;
b) determining if the one or more cardiac progenitor cells respond to the neuregulin peptide or functional variant or fragment by suppressing conversion of the cells into fibroblasts and myofibroblasts, as compared to conversion of one or more control cardiac progenitor cells not exposed to the neuregulin peptide or functional variant or fragment; and
c) administering a therapeutically effective amount of the neuregulin peptide or functional variant or fragment to the subject if conversion of the one or more cardiac progenitor cells is suppressed.

51. The method of claim 50, wherein the one or more cardiac progenitor cells are isolated from the subject during cardiac surgery or cardiac catherization.

52. The method of claim 50, wherein the one or more cardiac progenitor cells are from a left ventricle free wall or a vascular or perivascular region in the heart of the subject.

53. The method of claim 50, wherein the one or more cardiac progenitor cells express stem cell antigen-1 (sca-1).

54. The method of claim 50, wherein the neuregulin peptide or functional variant or fragment is administered intravenously or subcutaneously.

55. The method of claim 50, wherein the neuregulin peptide or functional variant or fragment is administered simultaneously, sequentially, continuously, or intermittently with a therapeutically effective amount of a chemotherapeutic agent, one or more embryonic stem cells, and/or one or more cardiac progenitor cells.

56. The method of claim 55, wherein the chemotherapeutic agent is azacitidine.

57. The method of claim 55, wherein the one or more cardiac progenitor cells are from the subject.

58. The method of claim 50, wherein the neuregulin peptide or functional variant or fragment comprises an NRG-1 peptide or a functional variant or fragment thereof, an NRG-2 peptide or a functional variant or fragment thereof, and/or an epidermal growth factor (EGF)-like domain.

59. The method of claim 58, wherein the NRG-1 peptide or functional variant or fragment comprises an NRG-1β peptide and/or a GGF2 peptide.

60. The method of claim 59, wherein the NRG-1β peptide comprises the amino acid sequence of SEQ ID NO: 19, SEQ ID NO: 20, or a functional variant or fragment of SEQ ID NO: 19 or SEQ ID NO: 20.

61. The method of claim 59, wherein the GGF2 peptide comprises the amino acid sequence of any one of SEQ ID NOs: 1-10, or a functional variant or fragment of any one of SEQ ID NOs: 1-10.

62. The method of claim 58, wherein the NRG-2 peptide or functional variant or fragment comprises the amino acid sequence of SEQ ID NO: 21, SEQ ID NO: 22, or a functional variant or fragment of SEQ ID NO: 21 or SEQ ID NO: 22.

63. The method of claim 58, wherein the EGF-like domain comprises the amino acid sequence of any one of SEQ ID NOs: 13-18, or a functional variant or fragment of any one of SEQ ID NOs: 13-18.

64. The method of claim 50, wherein the cardiac injury results from coronary artery disease; heart failure; stroke; myocardial infarction; cardiomyopathy; hypertension; ischemic heart disease; atrial fibrillation; congenital heart disease; myocarditis; endocarditis; pericarditis; atherosclerosis; vascular disease; left ventricular systolic dysfunction; coronary bypass surgery; exposure to a cardiotoxic compound; thyroid disease; viral infection; gingivitis; drug abuse; alcohol abuse; and/or high blood cholesterol.

65. The method of claim 50, wherein the subject suffers or has suffered from a myocardial infarction and/or heart failure.


SEQUENCE LISTING (22 sequences)

SEQ ID NO: 1 - protein, 422 aa, Homo sapiens
MRWRRAPRRSGRPGPRAQRPGSAARSSPPLPLLPLLLLLGTAALAPGAAAGNEAAPAGASVCYSSPPSVGSVQELAQRAAVVIEGKVHPQRRQQGALDRKAAAAAGEAGAWGGDREPPAAGPRALGPPAEEPLLAANGTVPSWPTAPVPSAGEPGEEAPYLVKVHQVWAVKAGGLKKDSLLTVRLGTWGHPAFPSCGRLKEDSRYIFFMEPDANSTSRAPAAFRASFPPLETGRNLKKEVSRVLCKRCALPPQLKEMKSQESAAGSKLVLRCETSSEYSSLRFKWFKNGNELNRKNKPQNIKIQKKPGKSELRINKASLADSGEYMCKVISKLGNDSASANITIVESNATSTSTTGTSHLVKCAEKEKTFCVNGGECFMVKDLSNPSRYLCKCPNEFTGDRCQNYVMASFYSTSTPFLSLPE

SEQ ID NO: 2 - protein, 372 aa, Homo sapiens
GNEAAPAGASVCYSSPPSVGSVQELAQRAAVVIEGKVHPQRRQQGALDRKAAAAAGEAGAWGGDREPPAAGPRALGPPAEEPLLAANGTVPSWPTAPVPSAGEPGEEAPYLVKVHQVWAVKAGGLKKDSLLTVRLGTWGHPAFPSCGRLKEDSRYIFFMEPDANSTSRAPAAFRASFPPLETGRNLKKEVSRVLCKRCALPPQLKEMKSQESAAGSKLVLRCETSSEYSSLRFKWFKNGNELNRKNKPQNIKIQKKPGKSELRINKASLADSGEYMCKVISKLGNDSASANITIVESNATSTSTTGTSHLVKCAEKEKTFCVNGGECFMVKDLSNPSRYLCKCPNEFTGDRCQNYVMASFYSTSTPFLSLPE

SEQ ID NO: 3 - protein, 11 aa, Homo sapiens
VCLLTVAALPP

SEQ ID NO: 4 - protein, 15 aa, Homo sapiens
ASPVSVGSVQELVQR

SEQ ID NO: 5 - protein, 8 aa, Homo sapiens
WFVVIEGK

SEQ ID NO: 6 - protein, 9 aa, Homo sapiens
KVHEVWAAK

SEQ ID NO: 7 - protein, 6 aa, Homo sapiens
  MOD_RES (5)..(5): Any amino acid
DLLLXV

SEQ ID NO: 8 - protein, 13 aa, Homo sapiens
  MOD_RES (12)..(12): Any amino acid
LGAWGPPAFPVXY

SEQ ID NO: 9 - protein, 13 aa, Homo sapiens
  MOD_RES (10)..(10): Any amino acid
YIFFMEPEAXSSG

SEQ ID NO: 10 - protein, 13 aa, Homo sapiens
  MOD_RES (12)..(12): Any amino acid
KASLADSGEYMXK

SEQ ID NO: 11 - protein, 61 aa, Homo sapiens
SHLVKCAEKEKTFCVNGGECFMVKDLSNPSRYLCKCPNEFTGDRCQNYVMASFYKAEELYQ

SEQ ID NO: 12 - protein, 60 aa, Homo sapiens
SHLVKCAEKEKTFCVNGGECFMVKDLSNPSRYLCKCPNEFTGDRCQNYVMASFYKAEELY

SEQ ID NO: 13 - protein, 65 aa, Homo sapiens
SHLVKCAEKEKTFCVNGGECFMVKDLSNPSRYLCKCPNEFTGDRCQNYVMASFYSTSTPFLSLPE

SEQ ID NO: 14 - protein, 63 aa, Homo sapiens
SHLVKCAEKEKTFCVNGGECFMVKDLSNPSRYLCKCQPGFTGARCTENVPMKVQTQEKAEELY

SEQ ID NO: 15 - protein, 60 aa, Homo sapiens; identical to SEQ ID NO: 12

SEQ ID NO: 16 - protein, 69 aa, Homo sapiens
SHLVKCAEKEKTFCVNGGECFMVKDLSNPSRYLCKCPNEFTGDRCQNYVMASFYKHLGIEFMEKAEELY

SEQ ID NO: 17 - protein, 88 aa, Homo sapiens
SHLVKCAEKEKTFCVNGGECFMVKDLSNPSRYLCKCQPGFTGARCTENVPMKVQTQEKCPNEFTGDRCQNYVMASFYSTSTPFLSLPE

SEQ ID NO: 18 - protein, 83 aa, Homo sapiens
SHLVKCAEKEKTFCVNGGECFMVKDLSNPSRYLCKCQPGFTGARCTENVPMKVQTQEKCPNEFTGDRCQNYVMASFYKAEELY

SEQ ID NO: 19 - protein, 300 aa, Homo sapiens
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKHLGIEFMEQKRVLTITGICIALLVVGIMCVVAYCKTKKQRKKLHDRLRQSLRSERNNMMNIANGPHHPN

SEQ ID NO: 20 - protein, 53 aa, Homo sapiens
NSDSECPLSHDGYCLHDGVCMYIEALDKYACNCVVGYIGERCQYRDLKWWELR

SEQ ID NO: 21 - protein, 330 aa, Homo sapiens
MRRDPAPGFSMLLFGVSLACYSPSLKSVQDQAYKAPVVVEGKVQGLVPAGGSSSNSTREPPASGRVALVKVLDKWPLRSGGLQREQVISVGSCVPLERNQRYIFFLEPTEQPLVFKTAFAPLDTNGKNLKKEVGKILCTDCATRPKLKKMKSQTGQVGEKQSLKCEAAAGNPQPSYRWFKDGKELNRSRDIRIKYGNGRKNSRLQFNKVKVEDAGEYVCEAENILGKDTVRGRLYVNSVSTTLSSWSGHARKCNETAKSYCVNGGVCYYIEGINQLSCKCPNGFFGQRCLEKLPLRLYMPDPKQSVLWDTPGTGVSSSQWSTSPSTLDLN

SEQ ID NO: 22 - protein, 298 aa, Homo sapiens
MRRDPAPGFSMLLFGVSLACYSPSLKSVQDQAYKAPVVVEGKVQGLVPAGGSSSNSTREPPASGRVALVKVLDKWPLRSGGLQREQVISVGSCVPLERNQRYIFFLEPTEQPLVFKTAFAPLDTNGKNLKKEVGKILCTDCATRPKLKKMKSQTGQVGEKQSLKCEAAAGNPQPSYRWFKDGKELNRSRDIRIKYGNGRKNSRLQFNKVKVEDAGEYVCEAENILGKDTVRGRLYVNSVSTTLSSWSGHARKCNETAKSYCVNGGVCYYIEGINQLSCKCPVGYTGDRCQQFAMVNFS